(19) United States
(12) Patent Application Publication
(10) Pub. No.: US 2004/0092498 A1
(43) Pub. Date: May 13, 2004

(54) Substituted glycine derivatives for use as medicaments

(76) Inventors: David Blakemore, Sandwich (GB); Justin S. Bryans, Sandwich (GB); Wai-Lam Alex Chu, San Diego, CA (US); Graham N. Maw, Sandwich (GB); David J. Rawson, Sandwich (GB); Lisa R. Thompson, Sandwich (GB)

Correspondence Address: David R. Kurlandsky, Warner-Lambert Company LLC, 2800 Plymouth Road, Ann Arbor, MI, 48105, US

(21) Appl. No.: 10/640,520

(22) Filed: Aug. 13, 2003

Related U.S. Application Data
(60) Provisional application No. 60/413,856, filed on Sep. 25, 2002.

(30) Foreign Application Priority Data
Aug. 16, 2002 (GB) 0219153.4

(51) Int. Cl. A61K 31/397; A61K 31/44; A61K 31/40

(52) U.S. Cl. 514/210.01; 514/357; 514/408

(57) ABSTRACT

The compounds of formula (I) are substituted glycine derivatives useful in the treatment of epilepsy, faintness attacks, hypokinesia, cranial disorders, neurodegenerative disorders, depression, anxiety, panic, pain, arthritis, neuropathological disorders, sleep disorders, visceral pain disorders and gastrointestinal disorders. Processes for the preparation of the final products and intermediates useful in the process are included. Pharmaceutical compositions containing one or more of the compounds are also included.

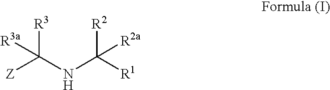

wherein R¹ is hydroxycarbonyl, a carboxylic acid biostere or prodrug thereof;

R³, R^{3a}, R² and R^{2a} are independently selected from H, C₁-C₆ alkyl, and C₁-C₆ alkoxy C₁-C₆ alkyl;

Z is;

(i) a C-linked, 5 membered heterocycloalky or heteroaryl substituted with C₁-C₆ alkyl or fused with C₃-C₈ cycloalkyl, 4-8 membered heterocycloalkyl, phenyl, or monocyclic heteroaryl, wherein the fused ring is optionally substituted with one or two substituents selected from the group consisting of halogen, C₁-C₆ alkyl, C₁-C₆ alkoxy, perfluoro C₁-C₆ alkyl, perfluoro C₁-C₆ alkoxy, cyano, C₁-C₆ alkyl amino, C₁-C₆ alkyl thio, C₃-C₈ cycloalkyl, 4-8 membered heterocycloalkyl, phenyl, and monocyclic heteroaryl; or

(ii) the group;

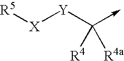

wherein R⁴ and R^{4a} are independently H, C₁-C₆ alkyl, C₁-C₆ alkoxy or C₁-C₆ alkoxy C₁-C₆ alkyl;

R⁵ is C₁-C₆ alkyl, C₃-C₁₂ cycloalkyl, 4-12 membered heterocycloalkyl, aryl or heteroaryl and R⁵ is optionally substituted with one or two substituents selected from the group consisting of halogen, C₁-C₆ alkyl, C₁-C₆ alkoxy, perfluoro C₁-C₆ alkyl, perfluoro C₁-C₆ alkoxy, cyano, C₁-C₆ alkyl amino, di-C₁-C₆ alkyl amino, amino C₁-C₆ alkyl, C₁-C₆ alkyl amino C₁-C₆ alkyl, di-C₁-C₆ alkyl amino C₁-C₆ alkyl, C₁-C₆ alkyl thio, C₃-C₈ cycloalkyl, 4-8 membered heterocycloalkyl, phenyl and monocyclic heteroaryl;

and either;

(i) Y is S, O, NH or CH₂ and X is a direct link or C₁-C₂ alkyl optionally substituted with C₁-C₆ alkyl or di-C₁-C₆ alkyl or 1-4 fluorine atoms; or

(ii) X is S, O, CH₂ or NH and Y is C₁-C₂ alkyl optionally substituted with C₁-C₆ alkyl or di-C₁-C₆ alkyl or 1-4 fluorine atoms.

This United States Utility Application claims the benefit of United Kingdom Application Number 0219153.4 filed on Aug. 16, 2002 and U.S. Provisional Application No. 60/413,856 filed on Sep. 25, 2002.

The invention relates to substituted glycine derivatives useful as pharmaceutical agents, to processes for their production, to pharmaceutical compositions containing them, and to their use for the treatment of the conditions set out below.

### BACKGROUND TO THE INVENTION

Gabapentin (Neurontin®) is an anti-convulsant agent that is useful in the treatment of epilepsy and has recently been shown to be a potential treatment for neurogenic pain. It is 1-(aminomethyl)-cyclohexylacetic acid of structural formula:

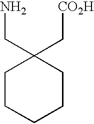

Gabapentin is one of a series of compounds of formula

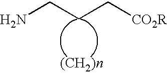

in which R is hydrogen or a lower alkyl radical and n is 4, 5, or 6. These compounds are described in U.S. Pat. No. 4,024,175 and its divisional U.S. Pat. No. 4,087,544. Their disclosed uses are: protection against thiosemicarbazide-induced cramp; protection against cardiazole cramp; the cerebral diseases, epilepsy, faintness attacks, hypokinesia, and cranial traumas; and improvement in cerebral functions. The compounds are useful in geriatric patients. The disclosures of the above two patents are hereby incorporated by reference.

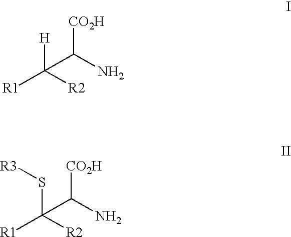

WO 0230871 describes compounds of the type I and WO 0222568 describes compounds of the type II. The compounds also have affinity for the gabapentin binding site and preferably have physiological activities similar to gabapentin particularly with respect to analgesia.

Some compounds of the main structural types within the claims are exemplified in other publications, however their use is not related to that of the current disclosure. Examples include N-(2-phenoxy-ethyl)-alanine (Beilstein reg: 5407903, J. Med. Chem., 1974, 1337-8), N-(2-benzylsulphanyl-ethyl)-glycine (Beilstein reg. 2560287, J. Am. Chem. Soc., 1948, 1620) and N-(2-phenylsulphanyl-ethyl)-glycine (Beilstein reg. 6707704, J. Med. Chem., 1975, 50-53).

### SUMMARY OF THE INVENTION

The present invention provides substituted glycine derivatives and their prodrugs, pharmaceutically acceptable salts and solvates useful in the treatment of a variety of disorders including epilepsy, faintness attacks, hypokinesia, cranial disorders, neurodegenerative disorders, depression, anxiety, panic, pain, sleep disorders, osteoarthritis, rheumatoid arthritis, and neuropathological disorders. The compounds provided may also be useful in the treatment of visceral pain, functional bowel disorders such as gastro-esophageal reflux, dyspepsia, irritable bowel syndrome and functional abdominal pain syndrome, and inflammatory bowel diseases such as Crohn's disease, ileitis, and ulcerative colitis, and other types of visceral pain associated with dysmenorrhea, pelvic pain, cystitis and pancreatitis. They may also be used for the treatment of premenstrual syndrome.

Thus, the present invention provides the use of a compound of formula (I) or a pharmaceutically acceptable salt or solvate thereof, as a medicament;

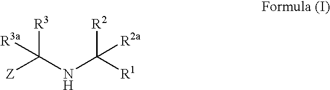

wherein R¹ is hydroxycarbonyl, a carboxylic acid biostere or prodrug thereof;

R³, R^{3a}, R² and R^{2a} are independently selected from H, C₁-C₆ alkyl, and C₁-C₆ alkoxy C₁-C₆ alkyl;

Z is either;

(i) a C-linked, 5-membered heterocycloalky or heteroaryl substituted with C₁-C₆ alkyl or fused with C₃-C₈ cycloalkyl, 4-8 membered heterocycloalkyl, phenyl, or monocyclic heteroaryl, wherein the fused ring is optionally substituted with one or two substituents selected from the group consisting of halogen, C₁-C₆ alkyl, C₁-C₆ alkoxy, perfluoro C₁-C₆ alkyl, perfluoro C₁-C₆ alkoxy, cyano, C₁-C₆ alkyl amino, C₁-C₆ alkyl thio, C₃-C₈ cycloalkyl, 4-8 membered heterocycloalkyl, phenyl, and monocyclic heteroaryl; or

(ii) the group;

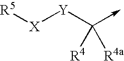

wherein R⁴ and R^{4a} are independently H, C₁-C₆ alkyl, C₁-C₆ alkoxy or C₁-C₆ alkoxy C₁-C₆ alkyl;

R⁵ is C₁-C₆ alkyl, C₃-C₁₂ cycloalkyl, 4-12 membered heterocycloalkyl, aryl or heteroaryl and R⁵ is optionally substituted with one or two substituents selected from the group consisting of halogen, C₁-C₆ alkyl, C₁-C₆ alkoxy, perfluoro C₁-C₆ alkyl, perfluoro C₁-C₆ alkoxy, cyano, C₁-C₆ alkyl amino, di-C₁-C₆ alkyl amino, amino C₁-C₆ alkyl, C₁-C₆ alkyl amino C₁-C₆ alkyl, di-C₁-C₆ alkyl amino C₁-C₆ alkyl, C₁-C₆ alkyl thio, C₃-C₈ cycloalkyl, 4-8 membered heterocycloalkyl, phenyl and monocyclic heteroaryl;

and either;

(i) Y is S, O, NH or CH₂ and X is a direct link or C₁-C₂ alkyl, optionally substituted with C₁-C₆ alkyl, di-C₁-C₆ alkyl or 1-4 fluorine atoms; or

(ii) X is S, O, CH₂ or NH and Y is C₁-C₂ alkyl, optionally substituted with C₁-C₆ alkyl or di-C₁-C₆ alkyl or 1-4 fluorine atoms.

According to formula (I), R¹ is suitably hydroxycarbonyl or a pro-drug comprising a C₁-C₆ ester, for example an ethyl or tert-butyl ester. R¹ is preferably hydroxycarbonyl.

According to formula (I), R², R^{2a}, R³ and R^{3a} independently are suitably C₁-C₆ alkyl or H and are preferably H.

According to formula (I), Z is suitably the group

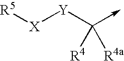

According to formula (I), R⁴ and R^{4a} are suitably C₁-C₆ alkyl or H, preferably H.

According to formula (I), R⁵ is suitably aryl or heteroaryl, optionally substituted with one or two substituents selected from the group consisting of halogen, C₁-C₆ alkyl, C₁-C₆ alkoxy, perfluoro C₁-C₆ alkyl, perfluoro C₁-C₆ alkoxy, C₁-C₆ alkyl thio and amino C₁-C₆ alkyl. R⁵ is preferably phenyl, naphthyl or isoquinolinyl and is most preferably phenyl or 7-isoquinolinyl, optionally substituted with one or two substituents selected from the group consisting of halogen, C₁-C₆ alkyl, C₁-C₆ alkoxy, perfluoro C₁-C₆ alkyl, perfluoro C₁-C₆ alkoxy, C₁-C₆ alkyl thio and amino C₁-C₆ alkyl.

According to formula (I), Y is suitably S, CH₂ or O and X is suitably a direct link or C₁-C₂ alkyl, for example CH₂, or X is suitably S, CH₂ or O and Y is suitably C₁-C₂ alkyl, for example CH₂.

Where Z is a substituted 5-membered heterocycloalky or heteroaryl, Z is preferably the group

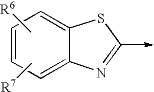

wherein R⁶ and R⁷ are independently H, halogen, C₁-C₆ alkyl, C₁-C₆ alkoxy, perfluoro C₁-C₆ alkyl, perfluoro C₁-C₆ alkoxy, cyano, C₁-C₆ alkyl amino, C₁-C₆ alkyl thio, C₃-C₈ cycloalkyl, 4-8 membered heterocycloalkyl, phenyl or monocyclic heteroaryl.

A preferred subgroup according to the present invention is represented by a compound of formula (II):

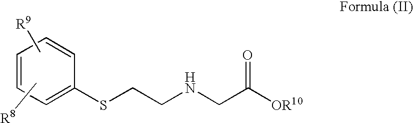

wherein R⁸ and R⁹ are independently H, halogen, C₁-C₆ alkyl, perfluoro C₁-C₆ alkyl, perfluoro C₁-C₆ alkoxy, C₁-C₆ alkyl thio or amino C₁-C₆ alkyl; and

R¹⁰ is H or C₁-C₆ alkyl, for example ethyl or tert-butyl.

According to formula (II), R⁸ and R⁹ are suitably positioned in the ortho- and para-positions and are suitably independently selected from H, bromo, chloro and aminomethyl.

A further preferred subgroup according to the present invention is represented by a compound of formula (III);

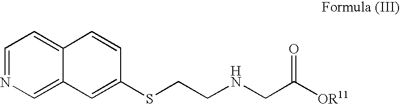

wherein R¹¹ is H or C₁-C₆ alkyl, for example tert-butyl.

Particularly preferred examples of the compounds of formula (I) are:

tert-Butyl ({2-[(4-bromophenyl)sulfanyl]ethyl}amino)acetate;

tert-Butyl ({2-[(4-chlorophenyl)sulfanyl]ethyl}amino)acetate;

tert-Butyl {[2-(2,4-dichlorophenoxy)ethyl]amino}acetate;

tert-Butyl ({2-[(4-chlorobenzyl)sulfanyl]ethyl}amino)acetate;

tert-Butyl {[2-(7-isoquinolinylsulfanyl)ethyl]amino}acetate;

({2-[(4-Chlorophenyl)sulfanyl]ethyl}amino)acetic acid;

({2-[(4-Bromophenyl)sulfanyl]ethyl}amino)acetic acid;

[(2-{[4-(Aminomethyl)phenyl]sulfanyl}ethyl)amino]acetic acid;

{[2-(2,4-Dichlorophenoxy)ethyl]amino}acetic acid;

({2-[(4-Chlorobenzyl)sulfanyl]ethyl}amino)acetic acid;

{[2-(7-Isoquinolinylsulfanyl)ethyl]amino}acetic acid;

Ethyl ({2-[(4-chlorophenyl)sulfanyl]ethyl}amino)acetate;

[2-(4-chloro-phenoxy)-propylamino]-acetic acid tert-butyl ester;

[2-(4-chloro-phenoxy)-propylamino]-acetic acid hydrochloride salt;

[2-(4-Methylsufanyl-phenylsufanyl)-ethylamino]-acetic acid tert-butyl ester;

[2-(4-Methylsufanyl-phenylsufanyl)-ethylamino]-acetic acid hydro-chloride salt;

(4-Phenyl-butylamino)-acetic acid methyl ester;

4-Phenylbutylamino acetic acid hydrochloride salt; and

[2-(3-Chloro-phenoxy)-butylamino]-acetic acid; dihydrochloride.

Particularly preferred compounds of the invention include those in which each variable in Formula (I) is selected from the suitable and/or preferred groups for each variable. Even more preferable compounds of the invention include those where each variable in Formula (I) is selected from the more preferred or most preferred groups for each variable.

It will be appreciated that certain compounds within the invention are novel and thereby these compounds, or a pharmaceutically acceptable salt, solvate, polymorph or pro-drug thereof, form a further aspect of the present invention. The invention also relates to pharmaceutical compositions comprising the compounds and their use as a medicament.

In the above definitions, halo means fluoro, chloro, bromo or iodo. Alkyl and alkoxy groups, containing the requisite number of carbon atoms, except where indicated, can be unbranched- or branched-chain. Examples of alkyl include methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy.

4-8 membered heterocycloalkyl when used herein refers to a single ring system containing at least one ring heteroatom independently selected from O, S and N. 4-12 membered heterocycloalkyl when used herein refers to a single ring or fused ring system containing at least one ring heteroatom independently selected from O, S and N. Thus a polycyclic fused ring system containing one or more carbocyclic fused saturated, partially unsaturated or aromatic rings is within the definition of 4-12 membered heterocycloalkyl so long as the system also contains at least one fused ring which contains at least one of the aforementioned heteroatoms. Suitable heterocycloalkyl groups include pyrrolidinyl, tetrahydrofuranyl, dihydrofuranyl, tetrahydropyranyl, pyranyl, thiopyranyl, aziridinyl, oxiranyl, methylenedioxyl, chromenyl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, chromanyl, etc.

Heteroaryl when used herein refers to a single aromatic ring or fused aromatic ring system containing at least one ring heteroatom independently selected from O, S and N. Thus, a polycyclic fused ring system containing one or more carbocyclic fused saturated, partially unsaturated or aromatic rings is within the definition of heteroaryl so long as the system also contains at least one fused aromatic ring which contains at least one of the aforementioned heteroatoms. Suitable heteroaryl groups include furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5, 6, 7, 8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl, benzoxazinyl, etc.

C₃-C₈ cycloalkyl as used herein refers to a single saturated or partially unsaturated carbocyclic ring system. C₃-C₁₂ cycloalkyl as used herein refers to a saturated or partially unsaturated single or fused carbocyclic ring system. Suitable cycloalkyl groups include cyclopropyl, cyclobutyl, cyclopentyl, cyclohexyl and cycloheptyl groups.

Aryl when used herein refers to phenyl or naphthyl.

Carboxylic acid biostere when used herein refers to a group functionally equivalent to a carboxylic acid. Suitable biosteres include tetrazole, oxazolidinone, sulfonic acid, sulfinic acid, phosphonic acid, phosphinic acid, hydantoin, pyrrolidione, 3-isoxazolyl, etc.

The present compounds can exist in unsolvated forms as well as solvated forms, including hydrated forms. In general, the solvated forms, including hydrated forms, which may contain isotopic substitutions (e.g. D2O, d6-acetone, d6-DMSO), are equivalent to unsolvated forms and are encompassed within the scope of the present invention.

Certain of the compounds of the present invention possess one or more chiral centers and each center may exist in the R or S configuration. The present invention includes all enantiomeric and epimeric forms as well as the appropriate mixtures thereof. Separation of diastereoisomers or cis and trans isomers may be achieved by conventional techniques, e.g. by fractional crystallisation, chromatography or H.P.L.C. of a stereoisomeric mixture of a compound of the invention or a suitable salt or derivative thereof. An individual enantiomer of a compound of the invention may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention also includes all suitable isotopic variations of a compound of the invention or a pharmaceutically acceptable salt thereof. An isotopic variation of a compound of the invention or a pharmaceutically acceptable salt thereof is defined as one in which at least one atom is replaced by an atom having the same atomic number but an atomic mass different from the atomic mass usually found in nature. Examples of isotopes that can be incorporated into compounds of the invention and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, 35S, ¹⁸F and ³⁶Cl, respectively. Certain isotopic variations of the compounds of the invention and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention and pharmaceutically acceptable salts thereof of this invention can generally be prepared by conventional procedures such as by the illustrative methods or by the preparations described in the Examples and Preparations hereafter using appropriate isotopic variations of suitable reagents.

Suitable pharmaceutically acceptable salts of the compounds of formula (I) can be salts of appropriate non-toxic inorganic or organic acids or bases. Suitable acid addition salts are the hydrochloride/chloride, hydrobromide/bromide, hydroiodide/iodide, sulphate, bisulphate, nitrate, phosphate, hydrogen phosphate, acetate, fumarate, aspartate, besylate, bicarbonate/carbonate, camsylate, D and L-lactate, D and L-tartrate, edisylate, mesylate, malonate, orotate, gluceptate, methylsulphate, stearate, glucuronate, 2-napsylate, tosylate, hibenzate, nicotinate, isethionate, malate, maleate, citrate, gluconate, succinate, saccharate, benzoate, esylate, and pamoate salts. Suitable base salts are formed from bases which form non-toxic salts and examples are the sodium, potassium, aluminium, calcium, magnesium, zinc, choline, diolamine, olamine, arginine, glycine, tromethamine, benzathine, lysine, meglumine and diethylamine salts. Salts with quaternary ammonium ions can also be prepared with, for example, the tetramethyl-ammonium ion. The compounds of the invention may also be formed as a zwitterion.

A suitable salt of compounds of the present invention is the hydrochloride salt. For a review on suitable salts see Berge et al, J. Pharm. Sci., 66, 1-19, 1977.

Also included within the present scope of the compounds of the invention are polymorphs thereof.

Prodrugs of the above compounds are included in the scope of the instant invention. The effectiveness of an orally administered drug is dependent upon the drug's efficient transport across the mucosal epithelium and its stability in entero-hepatic circulation. Drugs that are effective after parenteral administration but less effective orally, or whose plasma half-life is considered too short, may be chemically modified into a prodrug form. A prodrug is a drug which has been chemically modified and may be biologically inactive at its site of action, but which may be degraded or modified by one or more enzymatic or other in vivo processes to the parent bioactive form. This chemically modified drug, or prodrug, should have a different pharmacokinetic profile to the parent, enabling easier absorption across the mucosal epithelium, better salt formulation and/or solubility, improved systemic stability (for an increase in plasma half-life, for example). These chemical modifications may be

(1) Ester or amide derivatives which may be cleaved by, for example, esterases or lipases. For ester derivatives, the ester is derived from the carboxylic acid moiety of the drug molecule by known means. For amide derivatives, the amide may be derived from the carboxylic acid moiety or the amine moiety of the drug molecule by known means.

(2) Peptides which may be recognized by specific or nonspecific proteinases. A peptide may be coupled to the drug molecule via amide bond formation with the amine or carboxylic acid moiety of the drug molecule by known means.

(3) Derivatives that accumulate at a site of action through membrane selection of a prodrug form or modified prodrug form.

(4) Any combination of 1 to 3.

It will further be appreciated by those skilled in the art that certain moieties known to those skilled in the art as “pro-moieties”, for example as described in “Design of Prodrugs” by H Bundgaard (Elsevier) 1985, may be placed on appropriate functionalities when such functionalities are present in compounds of the invention also to form a “prodrug”. Further, certain compounds of the invention may act as prodrugs of other compounds of the invention. All protected derivatives, and prodrugs, of the compounds of the invention are included within the scope of the invention.

Research has shown that the oral absorption of certain drugs may be increased by the preparation of “soft” quaternary salts. The quaternary salt is termed a “soft” quaternary salt since, unlike normal quaternary salts, e.g., R—N⁺(CH₃)₃, it can release the active drug on hydrolysis. “Soft” quaternary salts have useful physical properties compared with the basic drug or its salts. Water solubility may be increased compared with other salts, such as the hydrochloride, but more important there may be an increased absorption of the drug from the intestine. Increased absorption is probably due to the fact that the “soft” quaternary salt has surfactant properties and is capable of forming micelles and unionized ion pairs with bile acids, etc., which are able to penetrate the intestinal epithelium more effectively. The prodrug, after absorption, is rapidly hydrolyzed with release of the active parent drug.

Aminoacyl-glycolic and -lactic esters are known as prodrugs of amino acids (Wermuth C. G., Chemistry and Industry, 1980:433-435). The carbonyl group of the amino acids can be esterified by known means. Prodrugs and soft drugs are known in the art (Palomino E., Drugs of the Future, 1990;15(4):361-368). The last two citations are hereby incorporated by reference.

The invention also relates to therapeutic use of the present compounds as agents for treating or relieving the symptoms of neurodegenerative disorders. Such neurodegenerative disorders include, for example, Alzheimer's disease, Huntington's disease, Parkinson's disease, and Amyotrophic Lateral Sclerosis. The present invention also covers treating neurodegenerative disorders termed acute brain injury. These include but are not limited to: stroke, head trauma, and asphyxia. Stroke refers to a cerebral vascular disease and may also be referred to as a cerebral vascular accident (CVA) and includes acute thromboembolic stroke. Stroke includes both focal and global ischemia. Also, included are transient cerebral ischemic attacks and other cerebral vascular problems accompanied by cerebral ischemia. These vascular disorders may occur in a patient undergoing carotid endarterectomy specifically or other cerebrovascular or vascular surgical procedures in general, or diagnostic vascular procedures including cerebral angiography and the like. Other incidents are head trauma, spinal cord trauma, or injury from general anoxia, hypoxia, hypoglycemia, hypotension as well as similar injuries seen during procedures from embole, hyperfusion, and hypoxia. The instant invention would be useful in a range of incidents, for example, during cardiac bypass surgery, in incidents of intracranial hemorrhage, in perinatal asphyxia, in cardiac arrest, and status epilepticus.

A skilled physician will be able to determine the appropriate situation in which subjects are susceptible to or at risk of, for example, stroke as well as suffering from stroke for administration by methods of the present invention.

The compounds of the present invention are useful for the general treatment of pain, particularly neuropathic pain. Physiological pain is an important protective mechanism designed to warn of danger from potentially injurious stimuli from the external environment. The system operates through a specific set of primary sensory neurones and is exclusively activated by noxious stimuli via peripheral transducing mechanisms (Millan 1999 Prog. Neurobio. 57: 1-164 for an integrative Review). These sensory fibres are known as nociceptors and are characterised by small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred after complex processing in the dorsal horn, either directly or via brain stem relay nuclei to the ventrobasal thalamus and then on to the cortex, where the sensation of pain is generated.

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies. Therefore pain can be divided into a number of different areas because of differing pathophysiology, these include nociceptive, inflammatory, neuropathic pain etc. It should be noted that some types of pain have multiple aetiologies and thus can be classified in more than one area, e.g. Back pain, Cancer pain can have nociceptive inflammatory and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmitted rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertebral discs or abnormalities of the lumber facet joints, sacroiliac joints, paraspinal muscles or the posterior longitudinal ligament

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, diabetic neuropathy, post herpetic neuralgia, back pain, cancer neuropathy, chemotherapy-induced neuropathy, HIV neuropathy, Phantom limb pain, Carpal Tunnel Syndrome, chronic alcoholism, hypothyroidism, trigeminal neuralgia, uremia, trauma-induced neuropathy, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The inflammatory process is a complex series of biochemical and cellular events activated in response to tissue injury or the presence of foreign substances, which result in swelling and pain (Levine and Taiwo 1994: Textbook of Pain 45-56). Arthritic pain makes up the majority of the inflammatory pain population. Rheumatoid disease is one of the commonest chronic inflammatory conditions in developed countries and rheumatoid arthritis (RA) is a common cause of disability. The exact aetiology of RA is unknown, but current hypotheses suggest that both genetic and microbiological factors may be important (Grennan & Jayson 1994 Textbook of Pain 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder 2002 Ann Pharmacother. 36: 679-686; McCarthy et al., 1994 Textbook of Pain 387-395). Most patients with OA seek medical attention because of pain. Arthritis has a significant impact on psychosocial and physical function and is known to be the leading cause of disability in later life. Other types of inflammatory pain include but are not limited to inflammatory bowel diseases (IBD),

Other types of pain include but are not limited to;

Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis.

Central pain or ‘thalamic pain’ as defined by pain caused by lesion or dysfunction of the nervous system including but not limited to central post-stroke pain, multiple sclerosis, spinal cord injury, Parkinson's disease and epilepsy.

Heart and vascular pain including but not limited to angina, myocardical infarction, mitral stenosis, pericarditis, Raynaud's phenomenon, sclerodoma, skeletal muscle ischemia.

Visceral pain, and gastrointestinal disorders. The viscera encompasses the organs of the abdominal cavity. These organs include the sex organs, spleen and part of the digestive system. Pain associated with the viscera may be neuropathic, nociceptive as well as inflammatory and can be divided into digestive visceral pain and non-digestive visceral pain. Commonly encountered gastrointestinal (GI) disorders include the functional bowel disorders (FBD) and the inflammatory bowel diseases (IBD). These GI disorders include a wide range of disease states that are currently only moderately controlled, including—for FBD, gastro-esophageal reflux, dyspepsia, the irritable bowel syndrome (IBS) and functional abdominal pain syndrome (FAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis. Few drugs are known to act selectively upon GI disorder-associated hypersensitivity (Farthing M. J. (1998) Drugs 56:11-21). Available treatments of pain fall into two main categories: (1) nonsteroidal anti-inflammatory drugs, used to treat mild pain, but whose therapeutic use is limited by GI adverse effects (gastric erosion, peptide ulcer formation, inflammation of the duodenum and colon); (2) morphine and related opioids, used to treat moderate to severe pain but whose therapeutic use is limited by undesirable side effects including constipation, respiratory depression, tolerance and abuse potential.

Head pain including but not limited to migraine, migraine with aura, migraine without aura, cluster headache, tension-type headache.

Orofacial pain including but not limited to dental pain, temporomandibular myofascial pain.

The compounds of the invention are also expected to be useful in the treatment of depression. Depression can be the result of organic disease, secondary to stress associated with personal loss, or idiopathic in origin. There is a strong tendency for familial occurrence of some forms of depression suggesting a mechanistic cause for at least some forms of depression. The diagnosis of depression is made primarily by quantification of alterations in patients' mood. These evaluations of mood are generally performed by a physician or quantified by a neuropsychologist using validated rating scales, such as the Hamilton Depression Rating Scale or the Brief Psychiatric Rating Scale. Numerous other scales have been developed to quantify and measure the degree of mood alterations in patients with depression, such as insomnia, difficulty with concentration, lack of energy, feelings of worthlessness, and guilt. The standards for diagnosis of depression as well as all psychiatric diagnoses are collected in the Diagnostic and Statistical Manual of Mental Disorders (Fourth Edition) referred to as the DSM-IV-R manual published by the American Psychiatric Association, 1994.

As a yet further aspect, there is provided the use of a compound of formula (I) in the manufacture of a medicament for the treatment of a disease selected from epilepsy, faintness attacks, hypokinesia, cranial disorders, neurodegenerative disorders, depression, anxiety, panic, pain, irritable bowel syndrome, sleep disorders, osteoarthritis, rheumatoid arthritis, neuropathological disorders, visceral pain, functional bowel disorders, inflammatory bowel diseases, pain associated with dysmenorrhea, pelvic pain, cystitis and pancreatitis.

As a alternative aspect, there is provided a method for treating a disease selected from epilepsy, faintness attacks, hypokinesia, cranial disorders, neurodegenerative disorders, depression, anxiety, panic, pain, irritable bowel syndrome, sleep disorders, osteoarthritis, rheumatoid arthritis, neuropathological disorders, visceral pain, functional bowel disorders, inflammatory bowel diseases, pain associated with dysmenorrhea, pelvic pain, cystitis and pancreatitis comprising administering a therapeutically effective amount of a compound of formula (I) to a mammal in need of said treatment.

The compounds of the instant invention may be administered in combination, either separately, simultaneously or sequentially, with one or more other pharmacologically active agents. Suitable agents, particularly for the treatment of pain, include:

(i) opioid analgesics, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, buprenorphine, butorphanol, nalbuphine and pentazocine;

(ii) Opioid antagonists, e.g. naloxone, naltrexone

(iii) nonsteroidal antiinflammatory drugs (NSAIDs), e.g. aspirin, diclofenac, difluinsal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and their pharmaceutically acceptable salts;

(iv) barbiturate sedatives, e.g. amobarbital, aprobarbital, butabarbital, butabital, mephobarbital, metharbital, methohexital, pentobarbital, phenobartital, secobarbital, talbutal, theamylal, thiopental and their pharmaceutically acceptable salts;

(v) benzodiazepines having a sedative action, e.g. chlordiazepoxide, clorazepate, diazepam, flurazepam, lorazepam, oxazepam, temazepam, triazolam and their pharmaceutically acceptable salts,

(vi) H₁ antagonists having a sedative action, e.g. diphenhydramine, pyrilamine, promethazine, chlorpheniramine, chlorcyclizine and their pharmaceutically acceptable salts;

(vii) miscellaneous sedatives such as glutethimide, meprobamate, methaqualone, dichloralphenazone and their pharmaceutically acceptable salts;

(viii) skeletal muscle relaxants, e.g. baclofen, tolperisone, carisoprodol, chlorzoxazone, cyclobenzaprine, methocarbamol, orphrenadine and their pharmaceutically acceptable salts,

(ix) NMDA receptor antagonists, e.g. dextromethorphan ((+)-3-hydroxy-N-methylmorphinan) and its metabolite dextrorphan ((+)-3-hydroxy-N-methylmorphinan), ketamine, memantine, pyrroloquinoline quinone and cis-4-(phosphonomethyl)-2-piperidinecarboxylic acid and their pharmaceutically acceptable salts;

(x) alpha-adrenergic active compounds, e.g. doxazosin, tamsulosin, clonidine and 4-amino-6,7-dimethoxy-2-(5-methanesulfonamido-1,2,3,4-tetrahydroisoquinol-2-yl)-5-(2-pyridyl) quinazoline;

(xi) tricyclic antidepressants, e.g. desipramine, imipramine, amytriptiline and nortriptiline;

(xii) anticonvulsants, e.g. carbamazepine, valproate, lamotrigine;

(xiii) serotonin reuptake inhibitors, e.g. fluoxetine, paroxetine, citalopram and sertraline;

(xiv) mixed serotonin-noradrenaline reuptake inhibitors, e.g. milnacipran, venlafaxine and duloxetine;

(xv) noradrenaline reuptake inhibitors, e.g. reboxetine;

(xvi) Tachykinin (NK) antagonists, particularly Nk-3, NK-2 and NK-1 e.g. antagonists, (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1 R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S)

(xvii) Muscarinic antagonists, e.g oxybutin, tolterodine, propiverine, tropsium chloride and darifenacin;

(xviii) COX-2 inhibitors, e.g. celecoxib, rofecoxib and valdecoxib;

(xix) Non-selective COX inhibitors (preferably with GI protection), e.g. nitroflurbiprofen (HCT-1026);

(xx) coal-tar analgesics, in particular, paracetamol;

(xxi) neuroleptics, such as droperidol;

(xxii) Vanilloid receptor agonists, e.g. resinferatoxin;

(xxiii) Beta-adrenergic compounds such as propranolol;

(xxiv) Local anaesthetics, such as mexiletine, lidocaine;

(xxv) Corticosteriods, such as dexamethasone

(xxvi) serotonin receptor agonists and antagonists;

(xxvii) cholinergic (nicotinic) analgesics; and

(xxviii) miscellaneous agents such as Tramadol®;

Combinations of the compounds of the present invention and other therapeutic agents may be administered separately, sequentially or simultaneously. Thus, the present invention extends to a kit comprising a compound of formula (I), one or more other therapeutic agents, such as those listed above, and a suitable container.

The biological activity of the compounds of the invention may be measured in a radioligand binding assay using [³H]gabapentin and the α₂δ subunit derived from porcine brain tissue (Gee N. S., Brown J. P., Dissanayake V. U. K., Offord J., Thurlow R., Woodruff G. N., J. Biol. Chem., 1996;271:5776-5879). Results may be expressed in terms of μM or nM α2δ binding affinity.

The compounds of the invention can be administered alone or in combination with other drugs but will generally be administered in an admixture with suitable pharmaceutical excipient(s), diluent(s) or carrier selected with regard to the intended route of administration and standard pharmaceutical practice. The term “excipient” is used herein to describe any ingredient other than the compound of the invention. If appropriate, auxiliaries can be added. Auxiliaries are preservatives, anti-oxidants, flavours or colourants. The compounds of the invention may be administered in a composition of the immediate-, delayed-, modified-, sustained-, pulsed- or controlled-release type.

The compounds of formula (I) can be administered, for example but not limited to the following routes: orally, buccally or sublingually in the form of tablets, capsules, multi- and nano-particulates, liquids, gels, films (incl. muco-adhesive), powders, ovules, elixers, lozenges (inc. liquid-filled), chews, solutions, suspensions and sprays. The compounds of formulae (I) may also be administered as osmotic dosage form, or in the form of a high energy dispersion or as coated particles or fast-dissolving, fast-disintegrating dosage form such as those described in Expert Opinion in Therapeutic Patents, 11(6), 981-986 by Liang and Chen (2001). The compounds of the formula (I) may be administered as crystalline or amorphous products. They may be obtained, for example as solid plugs, powders or films, by methods such as precipitation, crystallization, freeze drying, spray drying or evaporative drying. Microwave or radio frequency drying may also be used for this purpose. Suitable formulations of the compounds of formula (I) may be in hydrophilic or hydrophobic matrix, ion-exchange resin complex, coated or uncoated form and other types as described in U.S. Pat. No. 6,106,864 as desired. Such pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), mannitol, disintegrants such as sodium starch glycolate, crosscarmellose sodium and certain complex silicates, and granulation binders such as polyvinylpyrrolidone, hydroxypropylmethylcellulose (HPMC), triglycerides, hydroxypropylcellulose (HPC), bentonite sucrose, sorbitol, gelatin and acacia. Additionally, lubricating agents may be added to solid compositions, for example magnesium stearate, stearic acid, glyceryl behenate, PEG and talc or wetting agents, such as sodium lauryl sulphate or preservatives, anti-oxidants, flavours and colourants. Additionally, polymers such as carbohydrates, phospholipids and proteins may be included.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol or xylitol. The terms dispersing or dissolving as used herein to describe FDDFs are dependent upon the solubility of the drug substance used, i.e. where the drug substance is insoluble a fast dispersing dosage form can be prepared and where the drug substance is soluble a fast dissolving dosage form can be prepared.

The solid dosage forms, such as tablets are manufactured using standard processes known to a forumaltionchemist, for example, by direct compression, wet, dry or melt granulation, melt congealing or extrusion. The tablet cores which may be mono or multi-layer may be coated with appropriate overcoats known in the art.

Solid formulations for oral administration may be formulated to be immediate and/or modified release. Modified release formulations include delayed-, sustained-, pulsed-, controlled-, targeted and programmed release. Suitable modified release formulations for the purposes of the invention are described in U.S. Pat. No. 6,106,864. Details of other suitable release technologies such as high energy dispersions and osmotic and coated particles are to be found in Verma et al., Pharmaceutical Technology On-line, 25(2), 1-14 (2001).

Solid compositions of a similar type may also be employed as fillers in capsules such as gelatin, starch or HPMC capsules. Preferred excipients in this regard include lactose, starch, a cellulose, milk sugar or high molecular weight polyethylene glycols. Liquid compositions may be employed as fillers in soft or hard capsules, such as gelatin capsule, and typically comprise a carrier, for example water, ethanol, propylene glycol, methylcellulose or a suitable oil, and one or more emulsifying agents and/or suspending agents. For aqueous and oily suspensions, solutions, syrups and/or elixirs, the compounds of the invention may be combined with various sweetening or flavouring agents, colouring matter or dyes, with emulsifying and/or suspending agents and with diluents such as water, ethanol, propylene glycol, methylcellulose, alginic acid or sodium alginate, glycerin, oils, hydrocolloid agents and combinations thereof. Moreover, formulations containing these compounds and excipients may be presented as a dry product for reconstitution with water or other suitable vehicles before use.

Liquid form preparations include solutions, suspensions, syrups, elixirs and emulsions, for example, water or water propylene glycol solutions. For parenteral injection liquid preparations can be formulated in solution in aqueous polyethylene glycol solution. Aqueous solutions suitable for oral use can be prepared by dissolving the active component in water and adding suitable colorants, flavors, stabilizing and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

The compounds of the present invention can also be administered by injection, that is, intravenously, intramuscularly, intracutaneously, intraduodenally, or intraperitoneally, intra-arterially, intrathecally, intraventricularly, intraurethrally, intrasternally, intracranially, intraspinally or subcutaneously. Suitable devices for parenteral administration include needle (including microneedle) injectors, needle-free injectors, infusion or implant injection techniques. For such parenteral administration they are best used in the form of a sterile aqueous solution, suspension or emulsion (or system so that can include micelles) which may contain other substances known in the art, for example, enough salts or carbohydrates, such as glucose to make the solution isotonic with blood. The aqueous solutions should be suitably buffered (preferably to a pH of from 3 to 9), if necessary. For some forms of parenteral administration they may be used in the form of a sterile non-aqueous system such as fixed oils, including mono- or diglycerides, and fatty acids, including oleic acid. The preparation of suitable parenteral formulations under sterile conditions for example lyophilisation is readily accomplished by standard pharmaceutical techniques well-known to those skilled in the art. Alternatively, the active ingredient may be in a dried form to be used in conjunction with a suitable vehicle such as sterile, pyrogen-free water.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Thus, compounds of formula (I) may be formulated in a more solid form for administration as an implanted depot providing long-term release of the active compound.

Also, the compounds of the present invention can be administered intranasally or by inhalation. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomiser (preferably an atomiser using electrohydrodynamics to produce a fine mist) or nebuliser, with or without the use of a suitable propellant, e.g. dichlorodifluoromethane, trichlorofluoromethane, dichlorotetrafluoroethane, a hydrofluoroalkane such as 1,1,1,2-tetralfuoroethane (HFA 134A [trade mark]) or 1,1,1,2,3,3,3-heptafluoropropane (HFA 227EA [trade mark]), carbon dioxide, a further perfluorinated hydrocarbon such as Perflubron [trade mark] or other suitable gas.

The pressurised container, pump, spray, atomiser or nebuliser may contain a solution or suspension of the active compound, for example using a mixture of ethanol (optionally, aqueous ethanol) or a suitable agent for dispersing, solubilising or extending release and the propellant as the solvent, which may additionally contain a surfactant, such as sorbitan trioleate or an oligolactic acid. Capsules, blisters and cartridges (made, for example from gelatin or HPMC) for use in an inhaler or insufflator may be formulated to contain a powder mix of the compound of the invention, a suitable powder base such as lactose or starch and a performance modifier such as I-leucine, mannitol or magnesium stearate.

In the case of dry powder inhalers and aerosols, the dosage unit is determined by means of a valve which delivers a metered amount.

Prior to use in a dry powder formulation or suspension formulation for inhalation the compound of the invention is micronised to a size suitable for delivery by inhalation (typically considered as less than 5 microns). Micronisation may be achieved by any appropriate comminuting method, for example spiral jet milling, fluid bed jet milling, supercritical fluid processing to form nanoparticles, high pressure homogenisation or by spray drying.

A suitable solution formulation for use in an atomiser using electrohydrodynamics to produce a fine mist may contain from 1 μg to 10 mg of the compound of the invention per actuation and the actuation volume may vary from 1 μl to 100 μl. A typical formulation may comprise a compound of formula (I), propylene glycol, sterile water, ethanol and sodium chloride. Alternative solvents may be used in place of propylene glycol, for example glycerol or polyethylene glycol.

Formulations for inhaled/intranasal administration may be formulated to be immediate and/or modified release.

Alternatively, the compounds of the invention may be administered topically to the skin or mucosa, either dermally or transdermally, for example, in the form of a gel, hydrogel, lotion, solution, cream, ointment, dusting powder, dressing, foam, film, skin patch, wafers, implant, sponges, fibres, bandage, microemulsion and combinations thereof. Liposomes may also be used. For such applications, the compounds formula (I) can be suspended or dissolved in, for example, a mixture with one or more of the following: mineral oil, liquid petrolatum, white petrolatum, glycerin, propylene glycol, polyoxyethylene polyoxypropylene compound, emulsifying wax, fixed oils, including synthetic mono- or diglycerides, and fatty acids, including oleic acid, water, sorbitan monostearate, a polyethylene glycol, liquid paraffin, polysorbate 60, cetyl esters wax, cetearyl alcohol, 2-octyldodecanol, benzyl alcohol, alcohols such as ethanol. Alternatively, penetration enhancers may be used—see, for example J. Pharm. Sci., 88(10), 955-958 by Finnin and Morgan (October 1999). The following may also be used polymers, carbohydrates, proteins, phospholipids in the form of nanaparticles (such as niosomes or liposomes) or suspended or dissolved. In addition, they may be delivered using iontophoresis, electroporation, phonophoresis and sonophoresis.

Other means of topical administration include delivery by iontophoresis, electroporation, phonophoresis, sonophoresis and needle-free or microneedle injection.

Formulations for topical administration may be formulated to be immediate and/or modified release.

Alternatively, the compounds of the invention can be administered rectally, for example in the form of a suppository, pessary or enema. They may also be administered by vaginal route. For example, but not limited to the following presentations, these compositions may be prepared by mixing the drug with a suitable non-irritant excipients, such as cocoa butter, synthetic glyceride esters or polyethylene glycols, which are solid at ordinary temperatures, but liquefy and/or dissolve in the cavity to release the drug.

Formulations for rectal/vaginal administration may be formulated to be immediate and/or modified release.

The compounds of the invention may also be administered directly to the eye or ear. For ocular and aural administration, the compounds of formula (I) can be formulated as micronised suspensions or solutions in isotonic, pH adjusted, sterile saline. A polymer may be added such as crossed-linked polyacrylic acid, polyvinylalcohol, hyaluronic acid, a cellulosic polymer (e.g. hydroxypropylmethylcellulose, hydroxyethylcellulose or methyl cellulose), or a heteropolysaccharide polymer (e.g. gelan gum). Alternatively, they may be formulated in an ointment such as petrolatum or mineral oil, incorporated into biodegradable (e.g. absorbable gel sponges, collagen) or non-biodegradable (e.g. silicone) implants, wafers, drops, lenses or delivered via particulate or vesicular systems such as niosomes or liposomes. Formulations may be optionally combined with a preservative, such as benzalkonium chloride. In addition, they may be delivered using iontophoresis. Formulations for ocular/aural administration may be formulated to be immediate and/or modified release.

The compounds of the invention may also be used in combination with soluble macromolecular entities such as cyclodextrin or polyethylene glycol-containing polymers to improve their solubility, dissolution rate, taste-masking, bioavailability and/or stability. Drug-cyclodextrin complexes are generally useful for most dosage forms and administration routes. Both inclusion and non-inclusion complexes may be used. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The term ‘administered’ includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, lipsomes, immunoliposomes, lipofectin, cationic facial amphiphiles (CFAs) and combinations thereof. The routes for such delivery mechanisms include but are not limited to mucosal, nasal, oral, parenteral, gastrointestinal, topical or sublingual routes.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsules, tablet, cachet, or lozenge itself, or it can be the appropriate number of any of these in packaged form. The quantity of active component in a unit dose preparation may be varied or adjusted from 0.1 mg to 1 g according to the particular application and the potency of the active component. In medical use the drug may be administered three times daily as, for example, capsules of 100 or 300 mg. In therapeutic use, the compounds utilized in the pharmaceutical method of this invention are administered at the initial dosage of about 0.01 mg to about 100 mg/kg daily. A daily dose range of about 0.01 mg to about 100 mg/kg is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired.

The pharmaceutical composition according to the present invention can, if desired, also contain one or more other compatible therapeutic agents. In particular, the composition can be combined with any one or more compounds useful in the treatment of pain, such as those listed above. Thus, the present invention presents a pharmaceutical composition comprising a compound of formula (I), one or more other pharmacologically active agents and one or more pharmaceutically acceptable carriers.

It is to be appreciated that all references herein to treatment include curative, palliative and prophylactic treatment.

### General Methods

The compounds of formula (I) can be synthesized using the various methods set out below;

According to a first and second process, A and B, a compound of formula (I) may be prepared by reaction of a compound of formula (IV) with a compound of formula (V) or a compound of formula (VI) with a compound of formula (VII);

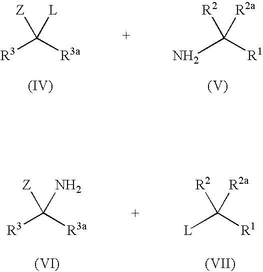

where L is a suitable leaving group such as halide, mesylate, tosylate or triflate leaving group or (═O), in the presence of a suitable base such as potassium carbonate in a suitable solvent such as dimethylformamide, or where L is (═O), under reductive amination using a suitable reducing agent such as NaCNBH₃ or Na(OAc)₃BH, a glycoxalate ester such as ethylglycoxalate, and a catalytic amount of an acid such as acetic acid in a solvent such as dichloromethane.

Alternatively, according to a third process C, a compound of formula (I) may be prepared by deprotection of a compound of formula (VIII);

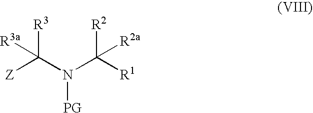

where PG is a suitable protecting group such as tert-butoxycarbonyl, by acid mediated hydrolysis of (VIII) using a suitable strong acid such as trifluoroacetic acid or hydrochloric acid in a suitable solvent such as dioxan or dichloromethane.

According to a fourth process D, where Z is

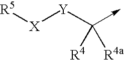

Y is S, O or NH and X is a direct link or C₁-C₂ alkyl, a compound of formula (I) may be prepared according to scheme 1.

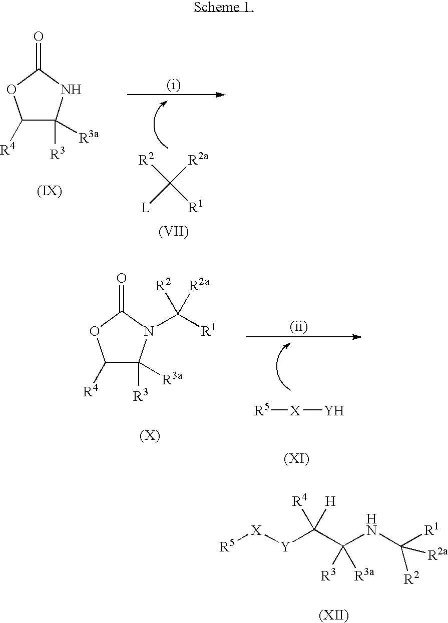

where L is a suitable leaving group such as halide, mesylate, tosylate or triflate leaving group.

Typical reaction conditions;

(i) Addition of an alkali metal salt such as potassium carbonate, or an alkali metal hydride such as sodium hydride to (IX) in a suitable solvent such as tetrahydrofuran at 0-25° C., followed by addition of (VII) and stirring at 0-100° C. for 2-24 h.

(ii) Addition of an alkali metal salt such as potassium tert-butoxide, a strong organic base such as 1,8-diazabicyclo[5.4.0]undec-7-ene (DBU), or an alkali metal hydride such as sodium hydride, to (X) in tetrahydrofuran or an alcoholic solvent such as tert-butanol, followed by addition of (XI) and reflux for 2-24h.

Alternatively, according to a fifth process E, where Z is

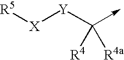

Y is S, O or NH, X is a direct link or C₁-C₂ alkyl and R^{4a} is H, a compound of formula (I) may be prepared according to scheme 2.

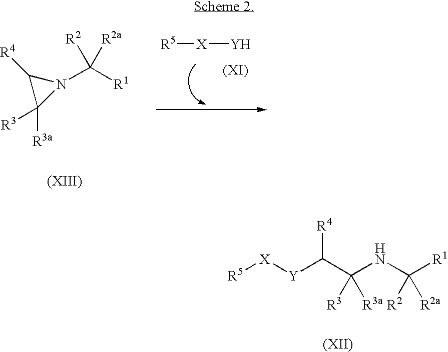

Typical reaction conditions;

Addition of an alkali metal salt such as potassium carbonate, or alkali metal hydride such as sodium hydride to (XIII) in a suitable solvent such as tertrahydrofuran, acetonitrile or dimethylformamide at 0-25° C., followed by addition of (XI) and stirring at 0-100° C. for 2-24 h.

Alternatively, according to a sixth process, F, where Z is

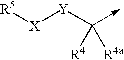

Y is S or O, X is a direct link and R⁴ is H, a compound of formula (I) may be prepared according to Scheme 3.

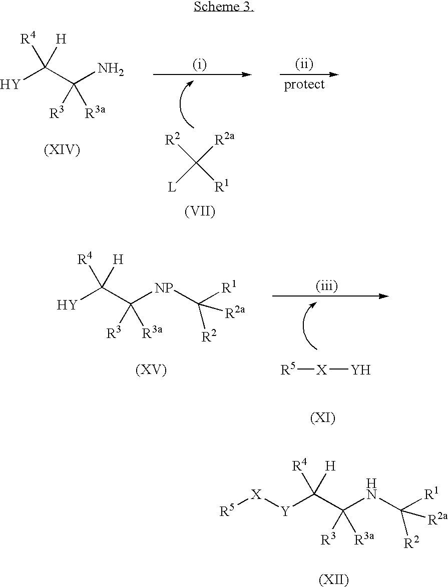

Where L is a suitable leaving group such as a halide, mesylate, tosylate or triflate group.

Typical reaction conditions;

(i) Addition of base such as triethylamine to (XIV) and (VII) in a suitable solvent such as diethyl ether at 25° C., stirring for 18 hours.

(ii) Protecting the nitrogen by the addition of e.g. Boc₂O, base such as sodium acetate in a suitable solvent such as dioxan at 25° C. for 4 hours to give (XV).

(iii) Addition of (XV), triphenylphosphine, an azodicarboxylate such as diisopropyl azodicarboxylate and (XI) in a suitable solvent such as tetrahydrofuran, stirring at 25° C. for 18 hours.

According to a seventh process, G, a compound of formula (I) may be prepared from a different compound of formula (I), using standard techniques well known in the art. For example, compounds of formula (I) where R¹ comprises a carboxylic acid group may be prepared from the compound of formula (I) where the corresponding position comprises a carboxylic ester group. Well known methods in the art may be employed to facilitate the transformation of an ester to an acid such as acid hydrolysis, using for example trifluoroacetic acid or hydrochloric acid, base mediated hydrolysis, using for example an alkali metal hydroxide such as sodium hydroxide, or hydrogenation with a suitable catalyst such as palladium on carbon.

Referring to the general methods above, it will be readily understood to the skilled person that where protecting groups are present, these will be generally interchangeable with other protecting groups of a similar nature, e.g. where an amine is described as being protected with a tert-butoxycarbonyl group, this may be readily interchanged with any suitable amine protecting group.

Compounds (IV)-(XV) may be prepared by literature methods known to the skilled person. It will be readily understood to the skilled person that particular steps in the general methods presented herein above may be suitably combined in any other manner not shown to provide a compound according to the present invention.

Thus, in summary, the invention provides:

(i) a process for the preparation of a compound of formula (I) or a pharmaceutically acceptable salt, solvate, polymorph or pro-drug thereof;

(ii) a pharmaceutical composition including a compound of formula (I) or a pharmaceutically acceptable salt, solvate, polymorph or pro-drug thereof, together with a pharmaceutically acceptable excipient, diluent or carrier;

(iii) a compound of formula (I) or a pharmaceutically acceptable salt, solvate, polymorph, pro-drug or composition thereof, for use as a medicament;

(iv) the use of a compound of formula (I) or of a pharmaceutically acceptable salt, solvate, polymorph, pro-drug or composition thereof, for the manufacture of a medicament for the treatment of any of the conditions mentioned herinbefore;

(v) the use of a compound of formula (I) or of a pharmaceutically acceptable salt, solvate, polymorph, pro-drug or composition thereof, for the manufacture of a medicament for the treatment of any of the conditions mentioned herinbefore;

(vi) a method of treatment of a mammal to treat any of the conditions mentioned herinbefore, including treating said mammal with an effective amount of a compound of formula (I) or with a pharmaceutically acceptable salt, solvate, polymorph, pro-drug or composition thereof;

(vii) a method for the treatment of any of the conditions mentioned herinbefore, which comprises administering to a patient in need of such treatment, either simultaneously, separately or sequentially, a combination of a compound of formula (I) and a further pain agent;

(viii) the use of a combination of a compound of formula (I) and a further therapeutic agent for the manufacture of a medicament for the treatment of any of the conditions mentioned herinbefore; and

(ix) a product containing a compound of formula (I) and a further therapeutic agent as a combined preparation for simultaneous, separate or sequential use in the treatment of any of the conditions mentioned herinbefore.

The present invention is illustrated by the following non-limiting examples and intermediates.

### EXAMPLE 1

### tert-Butyl ({2-[(4-bromophenyl)sulfanyl]ethyl}amino)acetate

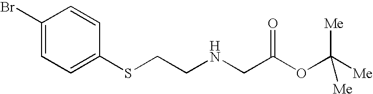

A stirred mixture of 4-bromothiophenol (10 g, 53 mmol) and potassium tert-butoxide (7.11 g, 63.5 mmol) in 2-methyl-2-propanol (250 ml) was heated to 60° C. for 30 minutes before the compound of Preparation 1 (12.8 g, 63.5 mmol) was added portionwise. The reaction mixture was heated up to 80° C. for 5 h and then concentrated to a thick syrup under vacuum. Water (250 ml) was added followed by 2M sodium hydroxide (10 ml) to pH=12. The organic phase was extracted with dichloromethane (3×250 ml), back washed with brine (150 ml), dried on magnesium sulphate and concentrated under vacuum yielding 16.6 g (90%) of light brown oil.

¹H-NMR (400 MHz, CDCl₃): δ=1.45 (s, 9H), 2.84 (t, 2H), 3.04 (t, 2H), 3.30 (s, 2H), 7.24 (d, 2H), 7.40 (d, 2H). MS (Electrospray): m/z [MH+] 348, [Mna+] 370, [2MH+] 715.

### EXAMPLE 2

### tert-Butyl ({2-[(4-chlorophenyl)sulfanyl]ethyl}amino)acetate

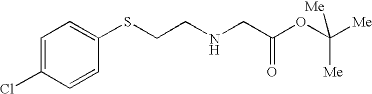

To 3.8 g (26.3 mmol) of 4-chlorothiophenol in 100 ml of tert-butanol at room temperature and under a nitrogen atmosphere was added 3.1 g (27.6 mmol) of potassium tert-butoxide in portions. The mixture was stirred for ten minutes and 5 g (24.8 mmol) of Preparation 1 was added and the mixture heated at reflux for 24 hours. The solvent was removed under reduced pressure and the yellow oily residue dissolved in diethyl ether (70 ml) and the title compound (7.1 g, 90%) isolated by precipitation as the hydrochloride salt by addition of 0.5M hydrochloric acid (50 ml), filtration and drying under reduced pressure.

¹H-NMR (400 MHz, CD₃OD) δ=1.51 (s, 9H), 3.2-3.28 (m, 4H), 3.90 (s, 2H), 7.36-7.38 (d, 2H), 7.44-7.47 (d, 2H) LRMS (electrospray): m/z [MH⁺] 302; [MNa⁺] 324

### EXAMPLE 3

### tert-Butyl {[2-(2,4-dichlorophenoxy)ethyl]amino}acetate

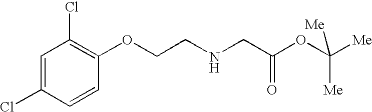

To 350 mg (1.44 mmol) of 2-(2,4-dichlorophenoxy)ethanamine and 0.2 ml (1.44 mmol) of triethylamine in 30 ml of dichloromethane stirring at room temperature under a nitrogen atmosphere was added 0.21 ml (1.44 mmol) aliquots, every 30 minutes, of tert-butyl bromoacetate until the starting material was completely consumed. The solvent was removed under reduced pressure and the residue purified by column chromatography on silica gel, eluting with dichloromethane:methanol:ammonia (95:5:0.5) to give the title compound (300 mg, 65%) as a colourless oil.

¹H-NMR (400 MHz, CDCl₃) δ=1.42-1.45 (s, 9H), 3.03-3.07 (t, 2H), 3.59 (s, 2H), 4.08-4.12 (t, 2H), 6.83-6.88 (d, 1H), 7.14-7.18 (m, 1H), 7.34-7.37 (m, 1H).

### EXAMPLE 4

### tert-Butyl ({2-[(4-chlorobenzyl)sulfanyl]ethyl}amino)acetate

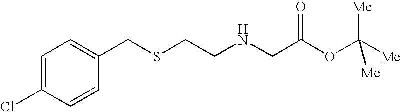

To a solution of 4-chlorobenzylmercaptan (370 mg, 2.33 mmol) in tert-butanol (10 ml) under nitrogen was added potassium tert-butoxide (140 mg, 1.2 mmol) and the reaction heated to 50° C. for 15 minutes. After cooling to room temperature, the oxazolidinone of Preparation 1 was added and the reaction heated to reflux for 18 hours. The reaction was cooled to room temperature and the solvent removed under reduced pressure. The residue was partitioned between water (10 ml) and dichloromethane (20 ml). The two phases were separated and the aqueous washed with dichloromethane (20 ml). The combined organic extracts were washed with saturated aqueous sodium chloride (15 ml), filtered, dried over magnesium sulphate and evaporated under reduced pressure. The crude product was purified by chromatography on silica (20 g) eluting with a solvent gradient of pentane:diethylether (2:1 by volume) changing to pentane:diethylether (1:1 by volume) to give the title compound as a colourless oil (246 mg, 62%)

¹H-NMR (400 MHz, CDCl₃): δ=1.39-1.52 (s, 9H), 1.95-2.11 (s, 1H), 2.52-2.59 (t, 2H), 2.76-2.78 (t, 2H), 3.25-3.30 (s, 2H), 3.67-3.71 (s, 2H), 7.23-7.30 (m, 4H). LRMS (electrospray): m/z [MNa⁺] 338

### EXAMPLE 5

### tert-Butyl {[2-(7-isoquinolinylsulfanyl)ethyl]amino}acetate

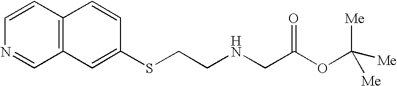

The thiol of Preparation 8 (975 mg, 6.05 mmol) was suspended in tert-butanol (30 ml) and potassium tert-butoxide (675 mg, 6.015 mmol) added. The mixture was heated to 60° C. for 30 minutes and the reaction cooled to R.T. The oxazolidinone of Preparation 1 was added and the reaction heated to reflux for 4 hours before leaving at room temperature overnight. The solvent was evaporated under reduced pressure and the residue dissolved in dichloromethane (25 ml). The solution was washed with water (10 ml), saturated aqueous sodium chloride solution (10 ml), dried over magnesium sulphate and evaporated under reduced pressure. Chromatography on silica (20 g) eluting with pentane:diethylether (2:1 by volume) yielded the title compound as a waxy white solid (317 mg, 20%)

1H-NMR (400 MHz, CDCl₃): δ=1.42-1.53 (s, 9H), 2.00-2.22 (s, 1H), 2.93-2.99 (t, 2H), 3.19-3.25 (t, 2H), 3.33-3.38 (s, 2H), 7.58-8.12 (m, 4H), 8.48-8.45 (m, 1H), 9.16-9.20 (s, 1H). LRMS (electrospray): m/z [MNa⁺] 341

### EXAMPLE 6

### ({2-[(4-Chlorophenyl)sulfanyl]ethyl}amino)acetic acid

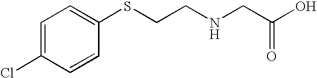

400 mg (1.18 mmol) of tert-butyl ({2-[(4-chlorophenyl)sulfanyl]ethyl}amino) acetate (Example 2) was dissolved/suspended in 10 ml of 4M anhydrous hydrogen chloride in dioxane solution and warmed to 50° C. for 1 hour. The solvent was removed under reduced pressure and the white solid residue recrystalized from isopropyl alcohol to give the title compound (250 mg, 86%).

¹H-NMR (400 MHz, D₂O) δ=3.12-3.13 (s, 4H), 3.64 (s, 2H), 7.26-7.29 (d, 2H), 7.29-7.32 (d, 2H) LRMS (electrospray): m/z [MH⁺] 246; [MNa⁺] 268; [MH⁻] 244 Microanalysis: Found: C, 42.50; H, 4.57; N, 4.79. C₁₀H₁₂ClNO₂S.HCl requires C, 42.56; H, 4.64; N, 4.96.

### EXAMPLE 7

### ({2-[(4-Bromoph nyl)sulfanyl] thyl}amino)ac tic acid

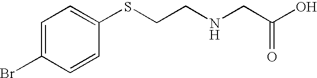

Using the method of Example 6, tert-butyl ({2-[(4-bromophenyl)sulfanyl]ethyl}amino)acetate (Example 1, 150 mg, 0.34 mmol) was hydrolysed yielding 60 mg (61%) of the title compound.

¹H-NMR (400 MHz, CD₃OD): δ=3.26 (s, 4H), 3.94 (s, 2H), 7.38 (d, 2H), 7.51 (d, 2H). MS (Electrospray): m/z [MH+] 291, [M−] 289. Microanalysis; Found: C, 36.46; H, 3.92; N, 4.25. C₁₁H₁₆NO₂S.1.1HCl requires C, 36.37; H, 4.0; N, 4.24.

### EXAMPLE 8

### [(2-{[4-(Aminomethyl)phenyl]sulfanyl}ethyl)amino]acetic acid

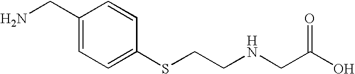

Using the method of Example 6, tert-butyl ({2-[(4-aminomethylphenyl)sulfanyl]ethyl}amino)acetate (30 mg, 0.076 mmol) was hydrolysed to the title compound (20 mg, 81%).

¹H-NMR (400 MHz, CD₃OD): δ=3.30 (m, 4H), 3.94 (s, 2H), 4.11 (s, 2H), 7.46 (d, 2H), 7.53 (d, 2H). MS (Electrospray): m/z [MH+] 241, [MNa+] 263, [2MH+] 481, [M−] 239. Microanalysis: Found: C, 40.93; H, 5.72; N, 8.28. C₁₁H₁₆N₂O₂S.2.3HCl requires C, 40.76; H, 5.69; N, 8.64.

### EXAMPLE 9

### {[2-(2,4-Dichlorophenoxy)ethyl]amino}acetic acid

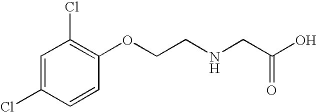

Using the method of Example 6, 300 mg (0.9 mmol) of tert-butyl {[2-(2,4-dichlorophenoxy)ethyl]amino}acetate (Example 3) was hydrolysed to give the title compound (58 mg, 22%) as a white solid.

¹H-NMR (400 MHz, D₆-DMSO) δ=3.39-3.43 (m, 2H), 3.97-3.98 (s, 2H), 4.36-4.40 (m, 2H), 7.19-7.23 (d, 1H), 7.37-7.42 (d, 1H), 7.59-7.60 (s, 1H) LRMS (thermospray): m/z [MH⁺] 264; [MNa⁺] 286; [MH⁻] 262 Microanalysis: Found: C, 40.10; H, 4.00; N, 4.59. C₁₀H₁₁NO₃Cl₂.HCl requires C, 39.96; H, 4.03; N, 4.66.

### EXAMPLE 10

### ({2-[(4-Chlorobenzyl)sulfanyl]ethyl}amino)acetic acid

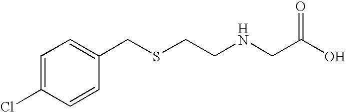

The method of Example 6 was used to hydrolyse the aminoester of Example 4 (215 mg, 0.68 mmol) to give the title compound as a white solid (138 mg, 68%)

¹H-NMR (400 MHz, CD₃OD): δ=2.71-2.79 (t, 2H), 3.21-3.32 (s, 2H), 3.79-3.88 (s, 2H), 3.91-3.98 (s, 2H), 4.86-4.93 (s, 3H), 7.29-7.41 (m, 4H). LRMS (electrospray): m/z [MH⁺] 260, [MNa+] 282, [M−1] 258. Microanalysis: Found: C, 44.56; H, 5.17; N; 4.70. C₁₁H₁₄ClNO₂S.HCl requires C, 44.690; H, 5.10; N, 4.73.

### EXAMPLE 11

### {[2-(7-Isoquinolinylsulfanyl)ethyl]amino}acetic acid

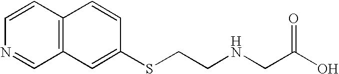

The aminoester of Example 5 (240 mg, 0.755 mmol) was dissolved in dichloromethane (6mi) and trifluoroacetic acid (2 ml) added. The reaction was stirred at room temperature for 5 hours and then the solvent removed under reduced pressure. The residue was triturated with diethylether to give a pale yellow solid half of which was dissolved in a minimum of water and purified by chromatography on MCl gel eluting with a gradient of water:acetonitrile (100:0) changing stepwise to water:acetonitrile (80:20). This yielded the title compound (30 mg, 15%) as a white solid.

¹H-NMR (400 MHz, CD₃OD): δ=3.26-3.33 (t, 2H), 3.39-3.45 (t, 2H), 3.51-3.54 (s, 2H), 4.77-4.83 (s, 3H), 7.78-7.82 (m, 2H), 7.91-7.94 (s, 1H), 8.13-8.15 (s, 1H), 8.41-8.43), 9.91-9.21 (s, 1H). LRMS (electrospray): m/z[MH⁺] 263, [MNa⁺] 285, [M−1] 261. Microanalysis: Found: 55.94; H, 5.71; N, 9.89. C₁₃H₁₄N₂O₂S.H₂O requires C, 55.70; H, 5.75; N, 9.99.

### EXAMPLE 12

### Ethyl ({2-[(4-chlorophenyl)sulfanyl]ethyl}amino)acetate

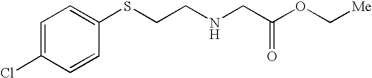

To 600 mg (1.77 mmol) of tert-butyl ({2-[(4-chlorophenyl)sulfanyl]ethyl}amino)acetate (Example 2) dissolved/suspended in 10 ml of ethanol was added 10 ml of 4M anhydrous hydrogen chloride in dioxane solution and the mixture heated to reflux for 30 minutes. The solvent was removed under reduced pressure and the white solid residue recrystalized from ethanol to give the title compound (423 mg, 88%).

¹H-NMR (400 MHz, D₂O) δ=1.08-1.12 (t, 3H), 3.13 (s, 4H), 3.80 (s, 2H), 4.09-4.12 (q, 2H), 7.23-7.30 (m, 4H) LRMS (electrospray): m/z [MH⁺] 274; [MNa⁺] 296 Microanalysis: Found: C, 46.36; H, 5.44; N, 4.41. C₁₂H₁₆ClNO₂S.HCl requires C, 46.46; H, 5.52; N, 4.51.

### EXAMPL 13

### [2-(4-chloro-phenoxy)-propylamino]-acetic acid tert-butyl ester

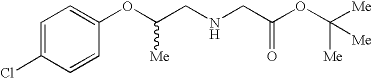

A mixture of 2-(4-chloro-phenoxy)-propionaldehyde¹ (0.07 g, 0.38 mmols), tert butyl glycine ester (0.054 ml, 0.40 mmols) and triethylamine (0.10 ml, 0.76 mmols) in dry dichloromethane (4 ml) was stirred at room temperature for 1 hour. Sodium triacetoxyborohydride (0.12 g, 0.57 mmols) was added portionwise and the reaction mixture stirred at room temperature for 48 hours. The reaction was quenched with saturated sodium hydrogen carbonate (15 ml) and stirred at room temperature for 15 mins. The aqueous was extracted with dichloromethane (3×10 ml), dried over magnesium sulfate, filtered and the solvent removed by evaporation under reduced pressure. The residue was dissolved in minimum dichloromethane and purified by flash chromatography on silica gel eluting with a solvent gradient of heptane:ethyl acetate (1:1) to give the title compound (0.036 g, 25%) as a colourless oil. ¹. Manetti, Dina; Romaneli, Maria Novella; Bartolini, Alessandro; Dei, Silivia; Gelardini, Carla; Gualtieri, Fulvio; Matucci, Rosanna; Scapecchi, Serena; Teodori, Elisabetta; Arch. Pharm (Weinheim, Ger.); 1996, 329(2), 105-11.

¹H-NMR (400 MHz, CD₃OD): δ=1.24 (d, 3H), 1.46 (s, 9H), 2.74 (m, 1H), 2.86 (m, 1H), 3.30 (s, 2H), 4.50 (m, 1H), 6.91 (d, 2H), 7.21 (d, 2H). LRMS (APCI): m/z [M+H]⁺ 300.

### EXAMPLE 14

### [2-(4-chloro-phenoxy)-propylamino]-acetic acid

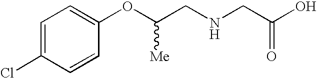

The above compound was synthesised using a method similar to Example 6, (0.032 g, 0.11 mmols) of [2-(4-chloro-phenoxy)-propylamino]-acetic acid tert butyl ester (Example 13) was hydrolysed to give the title compound (0.026 g, 89%) as a white solid.

¹H-NMR (400 MHz, CD₃OD): δ=1.32 (d, 3H), 3.36 (m, 2H), 3.96 (s, 2H), 4.80 (m, 1H), 7.01 (d, 2H), 7.31 (d, 2H). LRMS (Electrospray): m/z [M+H]⁺ 244. Microanalysis: Found: C, 46.96; H, 5.44; N, 4.94. C₁₁H₁₄NO₃Cl.HCl requires C, 47.16; H, 5.40; N, 5.00%.

### EXAMPLE 15

### [2-(4-Methylsufanyl-phenylsufanyl)-ethylamino]-acetic acid tert-butyl ester

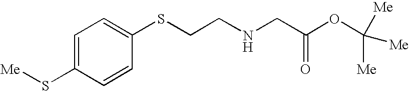

To a suspension of 2-(4-Methylsulfanyl-phenylsulfanyl)ethylamine hydrochloride salt (0.63 g, 2.69 mmols) in anhydrous tetrahydrofuran (20 ml) was added triethylamine (0.78 ml, 5.64 mmols) at 0° C. under nitrogen. The mixture was stirred for 30 mins. To this was added dropwise a solution of tert-butyl bromo acetate (0.42 ml, 2.82 mmols) in anhydrous tetrahydrofuran (10 ml). The mixture was warmed to room temperature and stirred fro 18 hours. The solvent was removed under reduced pressure and the residue partitioned between water (50 ml) and diethyl ether (30 ml). The aqueous was extracted with diethyl ether (2×30 ml), the combined organics dried over magnesium sulfate, filtered and the solvent removed under reduced pressure. The residue was purified by flash chromatography on silica gel eluting with a solvent gradient of heptane:ethyl acetate (1:1) to give the title compound (0.25 g, 29%) as a colourless oil.

¹H-NMR (400 MHz, CD₃OD): δ=1.44 (s, 9H), 2.45 (s, 3H), 2.75 (t, 2H), 3.01 (t, 2H), 3.20 (brs, 2H), 7.20 (d, 2H), 7.33 (d, 2H). LRMS (Electrospray): m/z [M+H]⁺ 314. Microanalysis: Found: C, 57.28; H, 7.38; N, 4.41. C₁₅H₂₃NO₂S₂ requires C, 57.47; H, 7.39; N, 4.47%.

### EXAMPLE 16

### [2-(4-Methylsufanyl-phenylsufanyl)-ethylamino]-acetic acid

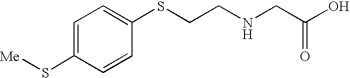

The above compound was synthesised using a method similar to Example 6, (0.20 g, 0.64 mmols) of [2-(4-Methylsufanyl-phenylsufanyl)-ethylamino]-acetic acid tert-butyl ester (Example 15) was hydrolysed to give the title compound (0.17 g, 91%) as a white solid.

¹H-NMR (400 MHz, CD₃OD): δ=2.47 (s, 3H), 3.21 (m, 4H), 3.92 (s, 2H), 7.25 (d, 2H), 7.41 (d, 2H). LRMS (Electrospray): m/z [M+H]⁺ 258. Microanalysis: Found: C, 44.92; H, 5.50; N, 4.67. C₁₁H₁₅NO₂S₂.HCl requires C, 44.96; H, 5.49; N, 4.77%.

### EXAMPLE 17

### (4-Phenyl-butylamino)-acetic acid²

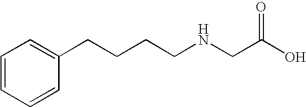

A mixture of the (4-phenylbutyl-amino)-acetic acid methyl ester (1.38 g, 6.25 mmol) in aqueous 6M HCl (70 ml) was stirred and heated to reflux at 120° C. for 18 hours. The mixture was cooled to room temperature and the solid filtered off and dried by evaporation under reduced pressure to give the title compound (1.26 g, 83%) as a white solid. ². Braun; Bayer; CHBEAM; Chem. Ber.; 60; 1927; 1259.

¹H-NMR (400 MHz, CD₃OD): δ=1.71 (m, 4H), 2.68 (m, 2H), 3.05 (m, 2H), 3.88 (m, 2H), 7.21 (m, 2H), 7.26 (m, 2H). LRMS (APCI): m/z [M+H]⁺ 208. Microanalysis: Found: C, 58.99; H, 7.41; N, 5.76. C₁₂H₁₇NO₂.HCl requires C, 59.14; H, 7.44; N, 5.75%.

### EXAMPLE 18

### [2-(3-Chloro-phenoxy)-butylamino]-acetic acid

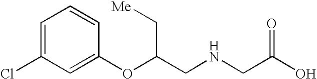

{tert-Butoxycarbonyl-[2-(3-chloro-phenoxy)-butyl]-amino}-acetic acid tert-butyl ester (140 mg, 0.33 mmol) stirred in trifluoroacetic acid (3 ml) and dichloromethane (3 ml) at room temperature for 18 hours. Solvent removed by evaporation under reduced pressure. The residue was taken into 1M hydrogen chloride (3 ml) and loaded onto Dowex® 50 WX8-200 resin. The resin was eluted with water:ammonia (95:5) to yield the product as a zwitterions which was stirred in hydrogen chloride in dichloromethane for 1 hour to yield the title compound as a white solid (33 mg, 38%).

¹H-NMR (400 MHz, CD₃OD) δ=0.99 (t, 3H), 1.79 (m 2H), 3.55 (s, 2H), 4.80 (m, 2H), 6.9 (m, 2H), 7.1 (s, 1H), 7.3 (t, 3H). LRMS (APCI): m/z [M+H]⁺ 258.

### EXAMPLE 19

### (4-Ph nyl-butylamino)-acetic acid methyl ster

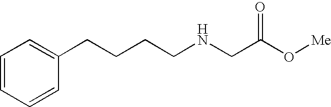

To a mixture of 4-phenylbutylamine (2.12 ml, 13.4 mmol) and triethylamine (1.84 ml, 13.4 mmol) in anhydrous tetrahydrofuran (30 ml), was added drop wise methyl bromoacetate (1.33 ml, 14.07 mmol) in anhydrous tetrahydrofuran (70 ml) at 0° C. The reaction mixture was warmed to room temperature and stirred for 18 hours. The solvent was removed by evaporation under reduced pressure. The residue was diluted with water (50 ml), extracted with diethyl ether (3×50 ml), dried over magnesium sulfate, filtered, and the solvent removed by evaporation under reduced pressure. The residue was dissolved in the minimum amount of dichloromethane and was purified by flash chromatography on silica gel eluting with solvent gradient of heptane:ethyl acetate (1:5) to give the title compound (1.58 g, 55%) as a colourless oil.

¹H-NMR (400 MHz, CDCl₃): δ=1.55 (m, 2H), 1.65 (m, 2H), 2.63 (m, 4H), 3.41 (s, 2H), 3.73 (s, 3H), 7.16 (m, 3H), 7.27 (m, 2H). LRMS (APCI): m/z [M+H]⁺ 222. Microanalysis: Found: C, 70.46; H, 8.66; N, 6.33. C₁₃H₁₉NO₂. Requires C, 70.56; H, 8.65; N, 6.33%.

### Preparation 1

### tert-Butyl (2-oxo-1,3-oxazolidin-3-yl)acetate

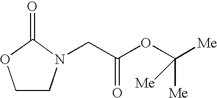

Sodium Hydride (60% dispersion in mineral oil, 10.1 g, 0.252 mol) was added portionwise to a stirred solution of 2-oxazolidinone (20 g, 0.23 mol) in dry tetrahydrofuran (250 ml) at 0° C. under nitrogen. After stirring for 30 minutes tert-butyl bromoacetate (50.8 ml, 0.344 mol) was added slowly and the reaction mixture was left heating to room temperature and stirred overnight. Water was then slowly added followed by ethyl acetate (250 ml). The aqueous phase was extracted and washed with ethyl acetate (50 ml). The combined organic phases were washed with brine (200 ml), dried over magnesium sulphate and concentrated under vacuum. The title compound crystallized on standing as a white solid (45 g, 97%).

¹H-NMR (400 MHz, CDCl₃): δ=1.46 (s, 9H), 3.68 (t, 2H), 3.91 (s, 2H), 4.36 (t, 2H).

### Preparation 2

### tert-Butyl [{2-[(4-bromophenyl)sulfanyl]ethyl}(tert-butoxycarbonyl)amino]acetate

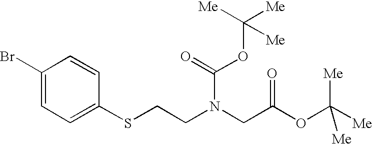

To a stirred solution of the compound of Example 1 (16 g, 46 mmol) in dichloromethane at 0° C. under nitrogen was added dropwise a solution of di-tert-butyl dicarbonate (11.1 g, 50.9 mmol) in dichloromethane (50 ml). After 15 h the reaction mixture was diluted with dichloromethane (150 ml) and washed successively with 1M sodium hydroxide (200 ml), ammonium chloride (saturated solution, 200 ml), 1M sodium hydroxide (200 ml), brine (300 ml) and dried over magnesium sulphate. The solvent was evaporated yielding a clear light brown oil that crystallized on standing (18.2 g, 89%).

¹H-NMR (400 MHz, CDCl₃): δ=1.44 (d, 9H), 1.47 (d, 9H), 3.07 (t, 1H), 3.13 (t, 1H), 3.40 (t, 1H), 3.45 (t, 1H), 3.81 (d, 2H), 7.24 (d, 2H), 7.39 (d, 2H). MS (Electrospray): [MNa+] 468, [2MNa+] 915.

### Preparation 3

### tert-Butyl ((tert-butoxycarbonyl){2-[(4-cyanophenyl)sulfanyl]ethyl}amino) acetate

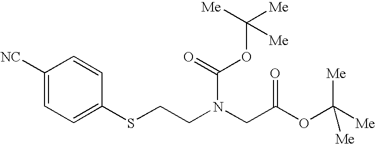

The compound of Preparation 2 (3 g, 6.7 mmol), zinc cyanide (787 mg, 6.7 mmol), tetrakis(triphenylphosphine)palladium (389 mg, 0.34 mmol) were mixed in degassed dry dimethylformamide (50ml) and heated up to 110° C. for 3 hours. The reaction mixture was then concentrated under vacuum to a thick syrup and water (30 ml) was added under vigorous stirring. The viscous oil solidified and after filtration, washing with water (3×50 ml) and drying under vacuum yielded 2.6 g (99%) of light brown solid.

¹H-NMR (400 MHz, CDCl₃): δ=1.45 (d, 9H), 1.48 (d, 9H), 3.21 (m, 2H), 3.49 (m, 2H), 3.85 (m, 2H), 7.35 (d, 1H), 7.40 (d, 1H), 7.53 (d, 2H). MS (Electrospray): m/z [MNa+] 415 [2MNa+] 807 [M−] 391.

### Preparation 4

### tert-Butyl [(2-{[4-(aminomethyl)phenyl]sulfanyl}ethyl)(tert-butoxycarbonyl)amino]acetate

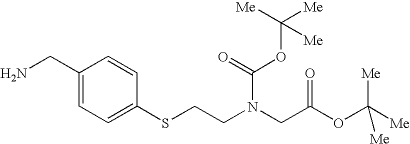

To a vigorously stirred slurry of the compound of Preparation 3 (300 mg, 0.76 mmol) and cobalt chloride (364 mg, 1.53 mmol) in methanol (10 ml) at −10° C. under nitrogen, was added sodium borohydride (291 mg, 7.6 mmol) portionwise. The black reaction mixture was left to warm to room temperature, stirred for 2.5 h, quenched with 5% aqueous HCl (50 ml) followed by water (15 ml) and 35% aqueous ammonia (40 ml), and extracted with ethyl acetate (3×300 ml). The title compound was isolated after chromatography on silica (eluent: gradiant from dichloromethane to dichloromethane/methanol 9/1) as a white solid (40 mg, 13%).

¹H-NMR (400 MHz, CDCl₃): δ=1.42 (d, 9H), 1.44 (d, 9H), 3.08 (m, 2H), 3.44 (m, 2H), 3.79 (d, 2H), 3.86 (s, 2H), 7.26 (d, 2H), 7.31 (d, 2H). MS (Electrospray): m/z [MH+] 397 [MNa+] 419 [2MH+]793. TLC: rf=0.1 in dichloromethane/methanol (9/1).

### Preparation 5

### 2,4-Dichloro-1-(2-chloroethoxy)benzene

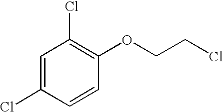

To 1 g (6.1 mmol) of 2,4-dichlorophenol in 10 ml of anhydrous dimethylformamide under a nitrogen atmosphere was added 270 mg (6.75 mmol) of 60% sodium hydride/oil dispersion. After stirring for 5 minutes, this solution was added dropwise to 10 ml of 1-bromo-2-chloroethane dissolved in 20 ml of anhydrous dimethylformamide and stirred for 24 hours at room temperature. The solvent was removed under reduced pressure reduced pressure and the residue dissolved in ethyl acetate (100 ml) and washed with water (1×50 ml). The organic layer was dried (MgSO₄), filtered and evaporated under reduced pressure. The resulting oil was purified by column chromatography on silica gel, eluting with pentane to give the title compound (1 g, 73%) as a colourless oil.

¹H-NMR (400 MHz, CDCl₃) δ=3.80-3.85 (t, 2H), 4.22-4.28 (t, 2H), 7.84-7.88 (d, 1H), 7.16-7.20 (d, 1H), 7.38 (s, 1H).

### Preparation 6

### Di(tert-butyl) 2-(2,4-dichlorophenoxy)ethylimidodicarbonate

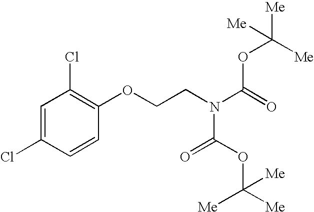

To 1 g (4.4 mmol) of 2,4-dichloro-1-(2-chloroethoxy)benzene in 10 ml of anhydrous dimethylformamide was added 1.06 g (4.4 mmol) of di(tert-butyl) imidodicarbonate sodium salt and heated at 90° C. for 16 hours. The solvent was removed under reduced pressure and the residue dissolved in ethyl acetate (100 ml) and washed with water (2×50 ml). The organic layer was dried (MgSO₄), filtered and evaporated under reduced pressure. The residue was purified by column chromatography on silica gel, eluting with a solvent gradient of pentane to 9:1 pentane:diethyl ether to give the title compound (700 mg) as a colourless oil.

¹H-NMR (400 MHz, CDCl₃) δ=1.49-1.51 (s, 18H), 4.02-4.05 (t, 2H), 4.15-4.18 (t, 2H), 6.84 (d, 1H), 7.15 (d, 1H), 7.34 (s, 1H).

### Preparation 7

### 2-(2,4-Dichlorophenoxy)ethanamine

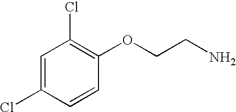

700 mg (1.7 mmol) of di(tert-butyl) 2-(2,4-dichlorophenoxy) ethylimidodicarbonate was dissolved in 15 ml of 4M anhydrous hydrogen chloride in dioxane solution and stirred for 2 hours at room temperature. The solvent was removed under reduced pressure and the residue was purified by trituration with ethyl acetate which upon filtration gave the title compound (350 mg, 100%) as a white solid.

¹H-NMR (400 MHz, D₂O) δ=3.33-3.38 (t, 2H), 4.18-4.25 (t, 2H), 6.95-7.00 (d, 1H), 7.18-7.23 (d, 1H), 7.40 (s, 1H) LRMS (thermospray): m/z [MH⁺] 206; [MNa⁺] 238.

### Preparation 8

### 7-Isoquinolinethiol

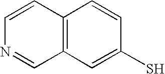

Triisopropylsilanethiol (6.49 g, 34.08 mmol) was added dropwise to a suspension of sodium hydride (1.36 g, 3.4 mmol, 60% dispersion in oil) in THF (100 ml) and the mixture stirred at room temperature for 20 minutes. Palladium(0) tetrakis(triphenylphosphine) (3.0 g, 2.5 mmol) was added followed by a solution of isoquinoline-7-trifluoromethanesuphonate (9.0 g, 32.5 mmol, Beilstein reg. No. 5439845) in THF (80 ml) and the reaction heated to reflux for 18 hours. After cooling to room temperature, the mixture was partitioned between ethyl acetate (150 ml) and water (75 ml). The phases were separated and the aqueos washed with ethyl acetate (2×50 ml). The combined organic extracts were backwashed with saturated aqueous sodium chloride solution (50 ml), dried over magnesium sulphate, filtered and the filtrate evaporated under reduced pressure. The crude material was purified by chromatography on silica (350 gm) eluting with a solvent gradient of dichloromethane (100:0 by volume) changing stepwise to dichloromethane:methanol (100:10 by volume) to give deprotected thiol. The thiol was dissolved in a minimum of hot ethanol and gravity filtered to remove a small amount of insoluble material. The filtrate was cooled in ice to yield the title compound (1.16 g, 21%) as a cream crystal line solid.

¹H-NMR (400 MHz, CDCl₃): δ=7.63 (d, 1H), 7.76-7.88 (m, 2H), 8.12 (s, 1H), 8.52 (d, 1H), 9.18 (s, 1H). LRMS (electrospray): [M−1] 160

### Preparation 9

### 2-(4-Methylsulfanyl-phenylsulfanyl)ethylamine hydrochloride salt

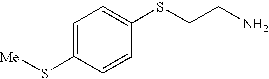

A mixture of 4-(methylthio)thiophenol (1 g, 6.34 mmols), sodium hydrogen carbonate (0.54 g, 6.40 mmols) and 2-bromo-ethylamine hydrobromide salt (1.48 g, 7.13 mmols) in absolute ethanol (20 ml) was stirred at reflux (90° C.) for 6 hours. The reaction mixture was cooled to room temperature and the solvent removed under reduced pressure. The solid was partitioned between 1M hydrochloric acid (50 ml) and diethyl ether (50 ml). The aqueous was washed with diethyl ether (2×50 ml). The aqueous evaporated under reduced pressure, washed with ethyl acetate (3×10 ml), the solid dried to give the title compound (0.66 g, 44%) as a white solid.

¹H-NMR (400 MHz, D₂O): δ=2.35 (s, 3H), 3.02 (m, 2H), 3.07 (m, 2H), 7.19 (d, 2H), 7.31 (d, 2H). LRMS (APCI): m/z [M+H]⁺ 200.

### Preparation 10

### (2-Hydroxybutylamino)-acetic acid tert-butyl ester

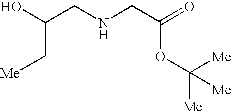

To a stirred solution of 1-amino-2-butanol (4.69 g, 5.26 mmol) in anhydrous diethyl ether (100 ml) was added triethylamine (1.88 ml, 13.49 mmol), followed by the drop wise addition of 1-bromo-t-butyl acetate (4.23 ml, 13.49 mmol) in diethyl ether (25 ml). The mixture was stirred at 0° C. for 3 hours, then warmed to room temperature and stirred for 18 hours. The mixture was washed with water (50 ml) and extracted with diethyl ether (2×50 ml). The organic layer was washed with brine (30 ml), dried over magnesium sulfate, filtered, and the solvent removed upon evaporation under reduced pressure to give the intermediate compound (3.07 g, 89%) as a colourless oil.

¹H-NMR (400 MHz, CD₃OD): δ=0.94 (t, 3H), 1.46 (2×s, 11H), 2.48 (dd, 1H), 2.62 (dd, 1H), 3.29 (d, 2H), 3.56 (m, 1H). LRMS (APCI): m/z [M+H]⁺ 204.

### Preparation 11

### [tert-butoxycarbonyl-(2-hydroxybutyl)-amino]-acetic acid tert-butyl ester

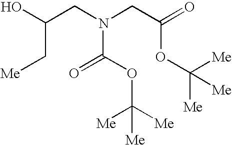

To a stirred solution of (2-Hydroxybutylamino)-acetic acid tert-butyl ester (2.74 g, 13.48 mmol) in dioxan (100 ml), was added di-tert-butyl dicarbonate (2.94 g, 13.48 mol), followed by the addition of sodium acetate (0.81 g, 13.48 mmol) in water (15 ml) and stirred at room temperature for 4 hours. The solvent was removed under by evaporation under reduced pressure. The residue was diluted with water (30 ml), extracted with ethyl acetate (3×50 ml), dried over magnesium sulfate and the solvent removed by evaporation under reduced pressure to give the title compound (2.24 g, 55%) as a colourless oil.

¹H-NMR (400 MHz, CD₃OD): δ=0.94 (t, 3H), 1.40 (m, 2H), 1.44 (3×s, 18H), 3.03 (m, 1H), 3.43 (m, 1H), 3.62 (m, 1H), 3.88 (d, 1H), 3.94 (d, 1H). LRMS (APCI): m/z [M+H]⁺ 304. Microanalysis: Found: C, 59.06; H, 9.70; N, 4.51. C₁₅H₂₉NO₅. Requires C, 59.38; H, 9.63; N, 4.62%.

### Preparation 12

### {tert-Butoxycarbonyl-[2-(3-chloro-phenoxy)-butyl]-amino}-acetic acid tert-butyl ester

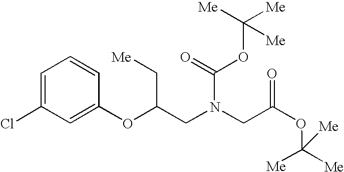

To a stirred solution of [tert-Butoxycarbonyl-(2-hydroxy-but yl)-amino]-acetic acid tert-butyl ester (300 mg, 0.99 mmol) in tetrahydrofuran (10 ml) was added triphenylphosphine (273 mg, 1.04 mmol) and 3-chloro phenol (133 mg, 1.04 mmol). Cooled to 0° C. and diisopropyl azodicarboxylate (239 mg, 1.04 mmol) in tetrahydrofuran (5 ml) was added drop-wise over 1 hour. The reaction mixture was warmed to room temperature and stirred for 18 hours. The solvent was removed under reduced pressure and the residue was taken into heptane:ethyl acetate (1:1) and stirred for 1 hour. The precipitate was filtered off and the filtrate concentrated, taken into ethyl acetate and washed with 2 N HCl (3×20 ml) and dried over magnesium sulphate. The solvent was removed by evaporation under reduced pressure and the residue was purified by flashmaster® column chromatography on silica eluting with heptane:ethyl acetate (15:1) to give the title compound (250 mg, 60%) as a clear oil.

¹H-NMR (400 MHz, CD₃OD): δ=0.98 (m, 3H), 1.45 (d, 18H), 1.65 (q, 2H), 3.32 (m, 1H), 3.65 (d, 1H), 3.85 (6, 2H), 4.48 (m, 1H), 6.85 (m, 1H), 6.90 (m, 2H), 7.2 (m, 1H). LRMS (ESI): m/z [M−H]⁺ 412. Microanalysis: Found: C, 60.68; H, 7.84; N, 3.44. C₂₁H₃₂ClNO₅ requires C, 60.93; H, 7.79; N, 3.43%.

### Pharmaceutical Composition Examples

In the following Examples, the active compound can be any compound of formula (I) and/or a pharmaceutically acceptable salt, solvate, or physiologically functional derivative thereof.

(i) Tablet Compositions

The following compositions A and B can be prepared by wet granulation of ingredients (a) to (c) and (a) to (d) with a solution of povidone, followed by addition of the magnesium stearate and compression.

| Composition A | mg/tablet | mg/tablet |
|---|---|---|
| (a) Active ingredient | 250 | 250 |
| (b) Lactose B.P. | 210 | 26 |
| (c) Sodium Starch Glycollate | 20 | 12 |
| (d) Povidone B.P. | 15 | 9 |
| (e) Magnesium Stearate | 5 | 3 |
| | 500 | 300 |

| Composition B | mg/tablet | mg/tablet |
|---|---|---|
| (a) Active ingredient | 250 | 250 |
| (b) Lactose | 150 | 150 |
| (c) Avicel PH 101 | 60 | 26 |
| (d) Sodium Starch Glycollate | 20 | 12 |
| (e) Povidone B.P. | 15 | 9 |
| (f) Magnesium Stearate | 5 | 3 |
| | 500 | 300 |

| Composition C | mg/tablet |
|---|---|
| Active ingredient | 100 |
| Lactose | 200 |
| Starch | 50 |
| Povidone | 5 |
| Magnesium Stearate | 4 |
| | 359 |

The following compositions D and E can be prepared by direct compression of the admixed ingredients. The lactose used in formulation E is of the direct compression type.

| Composition D | mg/tablet |
|---|---|
| Active ingredient | 250 |
| Magnesium Stearate | 4 |
| Pregelatinised Starch NF15 | 146 |
| | 400 |

| Composition E | mg/tablet |
|---|---|
| Active ingredient | 250 |
| Magnesium Stearate | 5 |
| Lactose | 145 |
| Avicel | 100 |
| | 500 |

| Composition F (Controlled release composition) | mg/tablet |
|---|---|
| (a) Active ingredient | 500 |
| (b) Hydroxypropylmethylcellulose | 112 |
| (Methocel K4M Premium) | |
| (c) Lactose B.P. | 53 |
| (d) Povidone B.P.C. | 28 |
| (e) Magnesium Stearate | 7 |
| | 700 |

The composition can be prepared by wet granulation of ingredients (a) to (c) with a solution of povidone, followed by addition of the magnesium stearate and compression.

Composition G (Enteric-Coated Tablet)

Enteric-coated tablets of Composition C can be prepared by coating the tablets with 25 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

Composition H (Enteric-Coated Controlled Release Tablet)

Enteric-coated tablets of Composition F can be prepared by coating the tablets with 50 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudgragit L). Except for Eudgragit L, these polymers should also include 10% (by weight of the quantity of polymer used) of a plasticizer to prevent membrane cracking during application or on storage. Suitable plasticizers include diethyl phthalate, tributyl citrate and triacetin.

(ii) Capsule Compositions

Composition A

Capsules can be prepared by admixing the ingredients of Composition D above and filling two-part hard gelatin capsules with the resulting mixture. Composition B (infra) may be prepared in a similar manner.

| Composition B | mg/capsule |
|---|---|
| (a) Active ingredient | 250 |
| (b) Lactose B.P. | 143 |
| (c) Sodium Starch Glycollate | 25 |
| (d) Magnesium Stearate | 2 |
| | 420 |

| Composition C | mg/capsule |
|---|---|
| (a) Active ingredient | 250 |
| (b) Macrogol 4000 BP | 350 |
| | 600 |

Capsules can be prepared by melting the Macrogol 4000 BP, dispersing the active ingredient in the melt and filling two-part hard gelatin capsules therewith.

| Composition D | mg/capsule |
|---|---|
| Active ingredient | 250 |
| Lecithin | 100 |
| Arachis Oil | 100 |
| | 450 |

Capsules can be prepared by dispersing the active ingredient in the lecithin and arachis oil and filling soft, elastic gelatin capsules with the dispersion.

| Composition E (Controlled release capsule) | mg/capsule |
|---|---|
| (a) Active ingredient | 250 |
| (b) Microcrystalline Cellulose | 125 |
| (c) Lactose BP | 125 |
| (d) Ethyl Cellulose | 13 |
| | 513 |

The controlled release capsule formulation can be prepared by extruding mixed ingredients (a) to (c) using an extruder, then spheronising and drying the extrudate. The dried pellets are coated with a release controlling membrane (d) and filled into two-part, hard gelatin capsules.

Composition F (Enteric capsule)

| | mg/capsule |
|---|---|
| (a) Active ingredient | 250 |
| (b) Microcrystalline Cellulose | 125 |
| (c) Lactose BP | 125 |
| (d) Cellulose Acetate Phthalate | 50 |
| (e) Diethyl Phthalat | 5 |
| | 555 |

The enteric capsule composition can be prepared by extruding mixed ingredients (a) to (c) using an extruder, then spheronising and drying the extrudate. The dried pellets are coated with an enteric membrane (d) containing a plasticizer (e) and filled into two-part, hard gelatin capsules.

Composition G (Enteric-Coated Controlled Release Capsule)

Enteric capsules of Composition E can be prepared by coating the controlled-release pellets with 50 mg/capsule of an enteric polymer such as cellulose acetate phthalate, polyvinylacetate phthalate, hydroxypropylmethylcellulose phthalate, or anionic polymers of methacrylic acid and methacrylic acid methyl ester (Eudragit L). Except for Eudragit L, these polymers should also include 10% (by weight of the quantity of polymer used) or a plasticizer to prevent membrane cracking during application or on storage. Suitable plasticizers include diethyl phthalate, tributyl citrate and triacetin.

(iii) Intravenous injection composition

| | | |
|---|---|---|
| Active ingredient | 0.200 | g |
| Sterile, pyrogen-free phosphate buffer (pH 9.0) to | 10 | ml |

The active ingredient is dissolved in most of the phosphate buffer at 35-40° C., then made up to volume and filtered through a sterile micropore filter into sterile 10 ml glass vials (Type 1) which are sealed with sterile closures and overseals.

(iv) Intramuscular injection composition

| | | |
|---|---|---|
| Active ingredient | 0.20 | g |
| Benzyl Alcohol | 0.10 | g |
| Glycofurol 75 | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (Type 1).

(v) Syrup composition

| | | |
|---|---|---|
| Active ingredient | 0.25 | g |
| Sorbitol Solution | 1.50 | g |
| Glycerol | 1.00 | g |
| Sodium Benzoate | 0.005 | g |
| Flavour | 0.0125 | ml |
| Purified Water q.s. to | 5.0 | ml |

The sodium benzoate is dissolved in a portion of the purified water and the sorbitol solution added. The active ingredient is added and dissolved. The resulting solution is mixed with the glycerol and then made up to the required volume with the purified water.

(vi) Suppository composition

| | mg/suppository |
|---|---|
| Active ingredient | 250 |
| Hard Fat, BP (Witepsol H15 - Dynamit NoBel) | 1770 |
| | 2020 |

One-fifth of the Witepsol H15 is melted in a steam-jacketed pan at 45° C. maximum. The active ingredient is sifted through a 200 lm sieve and added to the molten base with mixing, using a Silverson fitted with a cutting head, until a smooth dispersion is achieved. Maintaining the mixture at 45° C., the remaining Witepsol H15 is added to the suspension which is stirred to ensure a homogenous mix. The entire suspension is then passed through a 250 lm stainless steel screen and, with continuous stirring, allowed to cool to 40° C. At a temperature of 38-40° C., 2.02 g aliquots of the mixture are filled into suitable plastic moulds and the suppositories allowed to cool to room temperature.

(vii) Pessary composition

| | mg/pessary |
|---|---|
| Active ingredient (63 lm) | 250 |
| Anhydrous Dextrose | 380 |
| Potato Starch | 363 |
| Magnesium Stearate | 7 |
| | 1000 |

The above ingredients are mixed directly and pessaries prepared by compression of the resulting mixture.

(viii) Transdermal composition

| | | |
|---|---|---|
| Active ingredient | 200 | mg |
| Alcohol USP | 0.1 | ml |
| Hydroxyethyl cellulose | | |

The active ingredient and alcohol USP are gelled with hydroxyethyl cellulose and packed in a transdermal device with a surface area of 10 cm².

Biological Data

Compounds of the invention were tested in the radioligand binding assay described within and were found to have binding affinities as follows;

| | Activity |
|---|---|
| Exampl | (nM) |
| 9 | 1665 |
| 8 | 987 |
| 12 | 5406 |
| 6 | 198 |
| 10 | 507 |
| 11 | 71 |
| 7 | 59 |

1. A method of treating a disease selected from epilepsy, faintness attacks, hypokinesia, cranial disorders, neurodegenerative disorders, depression, anxiety, panic, pain, irritable bowel syndrome, sleep disorders, osteoarthritis, rheumatioid arthritis, neuropathological disorders, visceral pain, functional bowel disorders, inflammatory bowel diseases, pain associated with dysmenorrhea, pelvic pain, cystitis and pancreatitis in a mammal, comprising administering to said mammal a compound of formula (I) or a pharmaceutically acceptable salt or solvate thereof as a medicament; 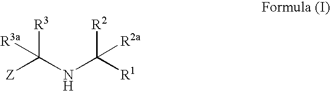
wherein R¹ is hydroxycarbonyl, a carboxylic acid biostere or prodrug thereof;
R³, R^{3a}, R² and R^{2a} are independently selected from H, C₁-C₆ alkyl, and C₁-C₆ alkoxy C₁-C₆ alkyl; and
Z is;
(i) a C-linked, 5-membered heterocycloalky or heteroaryl substituted with C₁-C₆ alkyl or fused with C₃-C₈ cycloalkyl, 4-8 membered heterocycloalkyl, phenyl, or monocyclic heteroaryl, wherein the fused ring is optionally substituted with one or two substituents selected from the group consisting of halogen, C₁-C₆ alkyl, C₁-C₆ alkoxy, perfluoro C₁-C₆ alkyl, perfluoro C₁-C₆ alkoxy, cyano, C₁-C₆ alkyl amino, C₁-C₆ alkyl thio, C₃-C₈ cycloalkyl, 4-8 membered heterocycloalkyl, phenyl and monocyclic heteroaryl; or
(ii) the group; 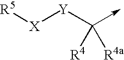
wherein R⁴ and R^{4a} are independently H, C₁-C₆ alkyl or C₁-C₆ alkoxy C₁-C₆ alkyl;
R⁵ is C₁-C₆ alkyl, C₃-C₁₂ cycloalkyl, 4-12 membered heterocycloalkyl, aryl or heteroaryl and R⁵ is optionally substituted with one or two substituents selected from the group consisting of halogen, C₁-C₆ alkyl, C₁-C₆ alkoxy, perfluoro C₁-C₆ alkyl, perfluoro C₁-C₆ alkoxy, cyano, C₁-C₆ alkyl amino, di-C₁-C₆ alkyl amino, amino C₁-C₆ alkyl, C₁-C₆ alkyl amino C₁-C₆ alkyl, di-C₁-C₆ alkyl amino C₁-C₆ alkyl, C₁-C₆ alkyl thio, C₃-C₈ cycloalkyl, 4-8 membered heterocycloalkyl, phenyl and monocyclic heteroaryl;
and either;
(i) Y is S, O, CH₂ or NH and X is a direct link or C₁-C₂ alkyl, optionally substituted with C₁-C₆ alkyl, di-C₁-C₆ alkyl or 1-4 fluorine atoms; or
(ii) X is S, O, CH₂ or NH and Y is C₁-C₂ alkyl, optionally substituted with C₁-C₆ alkyl, di-C₁-C₆ alkyl or 1-4 fluorine atoms.

2. A method according to claim 1, wherein Y is S, CH₂ or O and X is a direct link or C₁-C₂ alkyl.

3. A method according to claim 1, wherein X is S, CH₂ or O and Y is C₁-C₂ alkyl.

4. A method according to claim 1, wherein R², R^{2a}, R³, R^{3a}, R⁴ and R^{4a} are H or C₁-C₆ alkyl.

5. A method according to claim 1, wherein R⁵ is aryl or heteroaryl and is optionally substituted with one or two substituents selected from halogen, C₁-C₆ alkyl, C₁-C₆ alkoxy, perfluoro C₁-C₆ alkyl, perfluoro C₁-C₆ alkoxy, C₁-C₆ alkyl thio, and amino C₁-C₆ alkyl.

6. A method of treating a disease selected from epilepsy, faintness attacks, hypokinesia, cranial disorders, neurodegenerative disorders, depression, anxiety, panic, pain, irritable bowel syndrome, sleep disorders, osteoarthritis, rheumatoid arthritis, neuropathological disorders, visceral pain, functional bowel disorders, inflammatory bowel diseases, pain associated with dysmenorrhea, pelvic pain, cystitis and pancreatitis in a mammal, comprising administering to said mammal a compound of formula (II) or a pharmaceutically acceptable salt or solvate thereof; 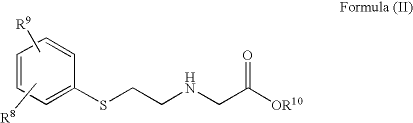
wherein R⁸ and R⁹ are independently H, halogen, C₁-C₆ alkyl, perfluoro C₁-C₆ alkyl, perfluoro C₁-C₆ alkoxy, C₁-C₆ alkyl thio or amino C₁-C₆ alkyl; and
R¹⁰ is H, or C₁-C₆ alkyl.

7. A method of treating a disease selected from epilepsy, faintness attacks, hypokinesia, cranial disorders, neurodegenerative disorders, depression, anxiety, panic, pain, irritable bowel syndrome, sleep disorders, osteoarthritis, rheumatoid arthritis, neuropathological disorders, visceral pain, functional bowel disorders, inflammatory bowel diseases, pain associated with dysmenorrhea, pelvic pain, cystitis and pancreatitis in a mammal, comprising administering to said mammal a compound of formula (III) or a pharmaceutically acceptable salt or solvate thereof; 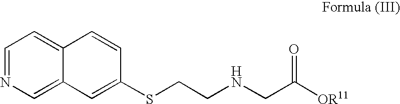
wherein R¹¹ is H or C₁-C₆ alkyl.

8. A method according to claim 1, wherein the compound is selected from the group consisting of;
tert-Butyl ({2-[(4-bromophenyl)sulfanyl]ethyl}amino)acetate;
tert-Butyl ({2-[(4-chlorophenyl)sulfanyl]ethyl}amino)acetate;
tert-Butyl {[2-(2,4-dichlorophenoxy)ethyl]amino}acetate;
tert-Butyl ({2-[(4-chlorobenzyl)sulfanyl]ethyl}amino)acetate;
tert-Butyl {[2-(7-isoquinolinylsulfanyl)ethyl]amino}acetate;
({2-[(4-Chlorophenyl)sulfanyl]ethyl}amino)acetic acid;
({2-[(4-Bromophenyl)sulfanyl]ethyl}amino)acetic acid;
[(2-{[4-(Aminomethyl)phenyl]sulfanyl}ethyl)amino]acetic acid;
{[2-(2,4-Dichlorophenoxy)ethyl]amino}acetic acid;
({2-[(4-Chlorobenzyl)sulfanyl]ethyl}amino)acetic acid;
{[2-(7-Isoquinolinylsulfanyl)ethyl]amino}acetic acid;
Ethyl ({2-[(4-chlorophenyl)sulfanyl]ethyl}amino)acetate;
[2-(4-chloro-phenoxy)-propylamino]-acetic acid tert-butyl ester;
[2-(4-chloro-phenoxy)-propylamino]-acetic acid hydrochloride salt;
[2-(4-Methylsufanyl-phenylsufanyl)-ethylamino]-acetic acid tert-butyl ester;
[2-(4-Methylsufanyl-phenylsufanyl)-ethylamino]-acetic acid hydrochloride salt;
(4-Phenyl-butylamino)-acetic acid methyl ester;
4-Phenylbutylamino acetic acid hydrochloride salt; and
[2-(3-Chloro-phenoxy)-butylamino]-acetic acid; dihydrochloride.

9. A pharmaceutical composition comprising a compound of formula (I) according to claim 1 and one or more pharmaceutically acceptable excipients and carriers.

10. A process for the preparation of a compound of formula (I); 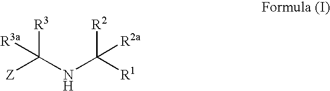
wherein R¹, R², R^{2a}, R³, R^{3a} and Z are as defined in claim 1, comprising:
(A) reaction of a compound of formula (IV) with a compound of formula (V), or a compound of formula (VI) with a compound of formula (VII); 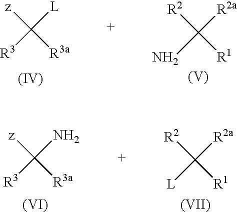
wherein L is a leaving group; or
(B) deprotection of a compound of formula (VIII); 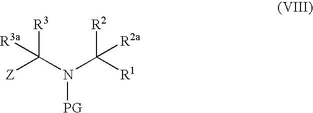
wherein PG is a suitable protecting group; or
(C) where X is a direct link or C₁-C₂ alkyl, ring opening of a compound of formula (X) or (XIII) by addition of a compound of formula (XI); 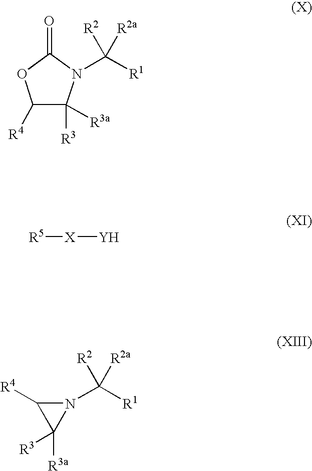
wherein R⁴, R^{4a}, R⁵, X and Y are as defined in claim 1.
